SEQUENCE LISTING (9 sequences)

SEQ ID NO: 1 - RNA, 20 nt, Artificial Sequence
  Synthetic Sequence
gaacucauggaccuuggcag

SEQ ID NO: 2 - RNA, 20 nt, Artificial Sequence
  Synthetic Sequence
gaacacaaggaccaaggcag

SEQ ID NO: 3 - RNA, 7 nt, Artificial Sequence
  Synthetic Sequence
aggaagu

SEQ ID NO: 4 - DNA, 20 bp, Artificial Sequence
  Synthetic Sequence
gaccccaaaatcagcgaaat

SEQ ID NO: 5 - DNA, 24 bp, Artificial Sequence
  Synthetic Sequence
tctggttactgccagttgaatctg

SEQ ID NO: 6 - DNA, 24 bp, Artificial Sequence
  Synthetic Sequence
accccgcattacgtttggtggacc

SEQ ID NO: 7 - DNA, 20 bp, Artificial Sequence
  Synthetic Sequence
gaccccaaaatcagcgaaat

SEQ ID NO: 8 - DNA, 24 bp, Artificial Sequence
  Synthetic Sequence
tctggttactgccagttgaatctg

SEQ ID NO: 9 - DNA, 24 bp, Artificial Sequence
  Synthetic Sequence
accccgcattacgtttggtggacc

(19) United States
(12) Patent Application Publication
(10) Pub. No.: US 2023/0201235 A1
(43) Pub. Date: Jun. 29, 2023

(54) INHIBITION OF NIDOVIRUSES THAT ENCODE NSP15

(71) Applicants: The University of Chicago, Chicago, IL (US); UChicago Argonne, LLC, Chicago, IL (US); Auburn University, Auburn, AL (US)

(72) Inventors: Youngchang Kim, Naperville, IL (US); Natalia Ivanovna Maltseva, Batavia, IL (US); Robert Jedrzejczak, Marengo, IL (US); Andrzej Joachimiak, Bolingbrook, IL (US); Jacek Wower, Auburn, AL (US); Glenn Randall, Chicago, IL (US)

(73) Assignees: The University of Chicago, Chicago, IL (US); UChicago Argonne, LLC, Chicago, IL (US); Auburn University, Auburn, AL (US)

(21) Appl. No.: 17/926,261

(22) PCT Filed: Mar. 11, 2021
(86) PCT No.: PCT/US2021/021913

§ 371 Date: Nov. 18, 2022

Related U.S. Application Data
(60) Provisional application No. 63/027,198, filed on May 19, 2020.

(51) Int. Cl. A61K 31/7072; A61K 31/7084; A61K 31/513; A61P 31/14; A61K 45/06

(52) U.S. Cl. CPC A61K 31/7072; A61K 31/7084; A61K 31/513; A61P 31/14; A61K 45/06; C07B 2200/13

(57) ABSTRACT

Provided is a method of treating a disease caused by a nidovirus that encodes Nsp15, such as a coronavirus, an arterivirus, or a torovirus, in a subject in need thereof comprising administering a therapeutically effective amount of an active agent selected from 3′-uridylic acid, 5′-uridylic acid, citrate, methacycline, meclocycline sulfosalicylate, mitoxantrone, epirubicin hydrochloride, daunorubicin hydrochloride, sorafenib, sunitinib malate, primaquine diphosphate, closantel, isopropyl ester of N4-hydroxycytidine, GpU dinucleotide or derivatives thereof, and tipiracil or N-substituted derivatives thereof. Further provided are a method of inhibiting an Nsp15 endoribonuclease of a nidovirus that encodes Nsp15 and compounds of formulas (I′) and (II′).

### CROSS-REFERENCE TO A RELATED APPLICATION

This patent application claims the benefit of U.S. Provisional Patent Application No. 63/027,198, filed May 19, 2020, which is incorporated by reference.

### STATEMENT REGARDING FEDERALLY SPONSORED RESEARCH OR DEVELOPMENT

This invention was made with Government support under contract number HHSN272201700060C awarded by the National Institute of Allergy and Infectious Diseases, National Institutes of Health. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,996 Byte ASCII (Text) file named “753202SequenceListing.txt”, created on Mar. 11, 2021.

### BACKGROUND OF THE INVENTION

SARS CoV-2 is a member of coronaviruses that are enveloped, non-segmented positive-sense RNA viruses from the order nidoviruses. The relatively large genome of these viruses contains a large replicase gene encompassing nonstructural proteins (Nsps), followed by structural and accessory genes. SARS CoV-2 has 15 Nsp proteins. Due to ribosomal frameshifting, the replicase gene encodes two ORFs, rep1a and rep1b, which are translated into two large polyproteins, pp1a and pp1ab. These polypeptides are processed by two viral proteases: 3C-like main protease (Mpro, encoded by Nsp5), and papain-like protease (PLpro, encoded within Nsp3). The cleavage yields 15 viral Nsps that assemble into a large membrane-bound replicase complex that exhibits multiple enzymatic activities. While several functions of Nsps have been linked to RNA replication and processing of subgenomic RNAs, the roles of some proteins are poorly understood or remain unknown.

Nsp15 is a nidoviral RNA uridylate-specific endoribonuclease (NendoU) carrying a C-terminal catalytic domain belonging to the EndoU family. EndoU enzymes are present in all kingdoms of life, where they play various biological functions associated with RNA processing. All characterized family members display an RNA endonuclease activity producing 2′,3′-cyclic phosphodiester and 5′-hydroxyl termini. The viral and eukaryotic enzymes act on both single- and double-stranded RNA and are specific for uridine. In viruses, the NendoU protein is conserved among coronaviruses, arteriviruses and toroviruses, but is absent in non-vertebrate-infecting representatives of the nidoviruses order: mesoniviruses and roniviruses. Nsp15 enzyme is essential in coronavirus biology.

Thus, there is a need for inhibiting Nsp15 activity to provide therapeutic interventions, particularly to treat diseases caused by nidoviruses that encode Nsp15.

### BRIEF SUMMARY OF THE INVENTION

The invention provides a method of treating a disease caused by a nidovirus that encodes Nsp15 in a subject in need thereof comprising administering to the subject a therapeutically effective amount of an active agent selected from 3′-uridylic acid (3′UMP), 5′-uridylic acid (5′UMP), citrate, a tetracycline antibiotic, an anthracycline antineoplastic agent, sorafenib, sunitinib malate, primaquine diphosphate, closantel, an isopropyl ester of N4-hydroxycytidine, (known as EIDD-1931/2801), a GpU dinucleotide of formula (I), and a compound of formula (II) or a pharmaceutically acceptable salt thereof.

The invention provides a method of inhibiting an Nsp15 endoribonuclease of a nidovirus that encodes Nsp15 comprising contacting the Nsp15 endoribonuclease with an effective amount of an active agent selected from 3′-uridylic acid (3′UMP), 5′-uridylic acid (5′UMP), citrate, a tetracycline antibiotic, an anthracycline antineoplastic agent, sorafenib, sunitinib malate, primaquine diphosphate, closantel, an isopropyl ester of N4-hydroxycytidine, a GpU dinucleotide of formula (I), and a compound of formula (II) or a pharmaceutically acceptable salt thereof.

### BRIEF DESCRIPTION OF THE DRAWINGS

FIG. 1 presents on the left a rendering of tipiracil bound to the active site of Nsp15, based on the determined X-ray crystal structure (FIG. 1A). The electron density map of tipiracil is shown on the right (FIG. 1B).

FIG. 2 presents on the left a rendering of 5′UMP bound to the active site of Nsp15, based on the determined X-ray crystal structure (FIG. 2A). The electron density map of 5′UMP is shown on the right (FIG. 2B).

FIG. 3 presents on the left a rendering of 3′UMP bound to the active site of Nsp15, based on the determined X-ray crystal structure (FIG. 3A). The electron density map of 3′UMP is shown on the right (FIG. 3B).

FIG. 4 presents on the left a rendering of GpU bound to the active site of Nsp15, based on the determined X-ray crystal structure (FIG. 4A). The electron density map of GpU is shown on the right (FIG. 4B).

FIG. 5 presents on the left a rendering of citrate bound to the active site of Nsp15, based on the determined X-ray crystal structure (FIG. 5A). The electron density map of citrate is shown on the right (FIG. 5B).

FIG. 6 presents on the left a rendering of uridine 2′,3′-vanadate (UV) bound to the active site of Nsp15, based on the determined X-ray crystal structure (FIG. 6A). The electron density map of UV is shown on the right (FIG. 6B).

FIG. 7 shows uridine-specific endoribonuclease activity of SARS-CoV-2 Nsp15. 5′-³²P-labeled RNA eicosamers were incubated with Nsp15 at 37° C. Reaction products were separated in a 20% polyacrylamide gel containing 7M urea. Digestion products of (FIGS. 7A, 7B, and 7C) GAACU₅CAUGG₁₀ACCUU₁₅GGCAG₂₀ (SEQ ID NO: 1) and (FIG. 7D) GAACA₅CAAGG₁₀ACCAA₁₅GGCAG₂₀ (SEQ ID NO: 2). U5-, U8- and U14/15-uridine-specific ladder. XC and BB mark final positions of the xylene cyanole (XC) and bromophenol blue (BB) dyes.

FIG. 8 shows the inhibition of SARS-CoV-2 Nsp15 endoribonuclease by tipiracil. RNA heptamers (5′AGGAAGU) (SEQ ID NO: 3), labeled at their 3′ ends with 3′,5′-[5′-³²P]bisphosphates to form octamers 5′ AGGAAGU³²pCp (SEQ ID NO: 3), were incubated at 30° C. with Nsp15 in the presence of tipiracil at 0, 1.85, 3.9, 7.8, 15.6, 31.25, 62.5, 125.0 and 250 μM (Lanes 1-9). Lane 10: minus enzyme control. Reaction products were separated in a 20% polyacrylamide gel containing 7 M urea. ³²P-labeled phosphates are marked with an asterisk. The continuous line represents the best fit for the Morrison equation (Copeland, R. A. Enzymes: A Practical Introduction to Structure. Mechanism. and Data Analysis. 2nd Edition. 305-317 (John Wiley & Sons, Inc., 2000)).

FIG. 9 demonstrates inhibition of SARS-CoV-2 coronavirus by tipiracil in whole cell assays. A549-hACE2 cells were pre-treated with tipiracil or carrier (0 mM) for 2 hrs and infected with CoV-2 at MOI (multiplicity of infection) 1. After 48 hrs, cells were harvested to check spike protein (FIG. 9A) and RNA expression (FIG. 9B). *P<0.05.

### DETAILED DESCRIPTION OF THE INVENTION

Without wishing to be bound by theory, Nsp15 has been proposed to possibly perform several functions. NendoU activity of Nsp15 may be responsible for protein interference with the innate immune response, facilitating evasion of host pattern recognition receptor MDA5. The enzyme has been shown to cleave 5′-polyuridines from negative-sense viral RNA, which is the product of polyA-templated RNA synthesis, reducing antiviral response. Nsp15 specifically recognizes U, and to a lesser extent C bases using a set of residues predicted from the EndoU and RNAse A, and it cleaves efficiently RNA containing U at the 3′ end of pU nucleotide. Nsp15 may degrade viral RNA to hide it from the host defenses.

Accordingly, the invention provides a method of treating a disease caused by a nidovirus that encodes Nsp15 (e.g., a coronavirus, an arterivirus, or a torovirus) in a subject in need thereof comprising administering to the subject a therapeutically effective amount of an active agent selected from 3′-uridylic acid (3′UMP), 5′-uridylic acid (5′UMP), citrate,

a tetracycline antibiotic of the formula

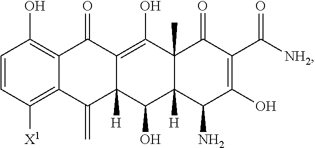

wherein X¹ is H or Cl and salts thereof,

an anthracycline antineoplastic agent with a core structure of

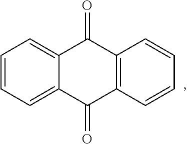 [SMILES: O=C1c2ccccc2C(=O)c2ccccc21]

sorafenib, sunitinib malate, primaquine diphosphate, closantel, an isopropyl ester of N4-hydroxycytidine,

a GpU dinucleotide of formula (I):

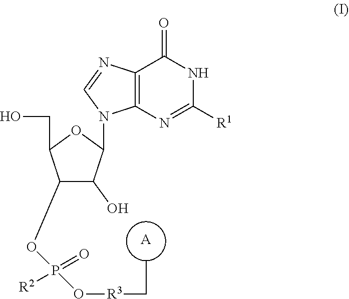

wherein

ring A is

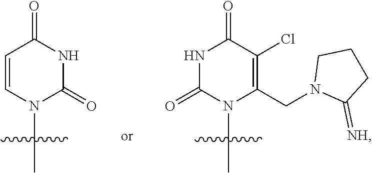 [SMILES: *n1ccc(=O)[nH]c1=O ; *n1c(CN2CCCC2=N)c(Cl)c(=O)[nH]c1=O]

R¹ is —NR⁴R⁵,

R² is O⁻M⁺ or —(CR⁶R⁷)ₙ—R⁸,

R³ is 1′,5′-ribosyl of the formula

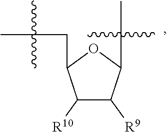

—CH₂CH₂CH₂CH₂—, —CH₂CH₂CH(CO₂H)CH₂—, or —CH₂CH(CO₂H)CH₂CH₂—,

R⁴ and R⁵ is the same or different and each is H or alkyl,

each instance of R⁶ and R⁷ is the same or different and each is H or alkyl,

R⁸ is alkyl, —OR¹¹, —SR¹¹, or —NR⁴R⁵, and

R⁹ and R¹⁰ are the same or different and each is —(CR⁶R⁷)ₘ—R⁸, or R⁹ and R¹⁰ together form —(CR⁶R⁷)ₒ—, and

R¹¹ is H or alkyl,

M⁺ is a counterion,

m and n are the same or different and each is 0 or an integer of 1 to 4, and

o is 3 or 4, and

a compound of formula (II):

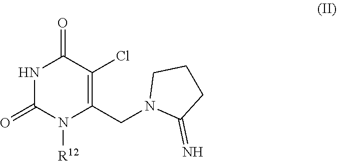

wherein

R¹² is H or —(CR¹³R¹⁴)ₙ—R¹⁵,

each instance of R¹³ and R¹⁴ is the same or different and each is H or alkyl,

R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,

R¹⁶ and R¹⁸ is the same or different and each is H or alkyl,

R¹⁷ is H, alkyl, OH, or SH,

X is O, S, or NR⁵, and

n is 0 or an integer of 1 to 4,

or a pharmaceutically acceptable salt thereof.

The invention further provides a method of inhibiting an Nsp15 endoribonuclease of a nidovirus that encodes Nsp15 (e.g., a coronavirus, an arterivirus, or a torovirus) comprising contacting the Nsp15 endoribonuclease with an effective amount of an active agent selected from 3′-uridylic acid (3′UMP), 5′-uridylic acid (5′UMP), citrate,

a tetracycline antibiotic of the formula

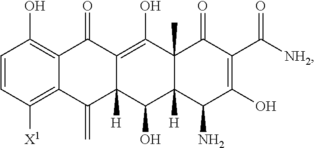

wherein X¹ is H or Cl and salts thereof,

an anthracycline antineoplastic agent with a core structure of

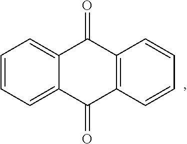 [SMILES: O=C1c2ccccc2C(=O)c2ccccc21]

sorafenib, sunitinib malate, primaquine diphosphate, closantel, isopropyl ester of N4-hydroxycytidine,

a GpU dinucleotide of formula (I):

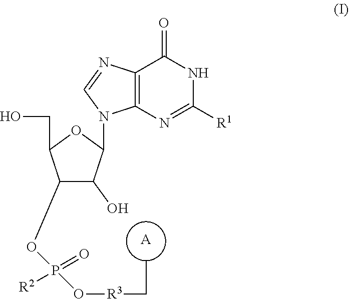

wherein

ring A is

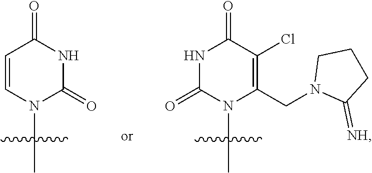 [SMILES: *n1ccc(=O)[nH]c1=O ; *n1c(CN2CCCC2=N)c(Cl)c(=O)[nH]c1=O]

R¹ is —NR⁴R⁵,

R² is O⁻M⁺ or —(CR⁶R⁷)ₙ—R⁸,

R³ is 1′,5′-ribosyl of the formula

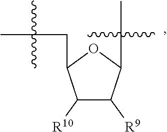

—CH₂CH₂CH₂CH₂—, —CH₂CH₂CH(CO₂H)CH₂—, or —CH₂CH(CO₂H)CH₂CH₂—,

R⁴ and R⁵ is the same or different and each is H or alkyl,

each instance of R⁶ and R⁷ is the same or different and each is H or alkyl,

R⁸ is alkyl, —OR¹¹, —SR¹¹, or —NR⁴R⁵, and

R⁹ and R¹⁰ are the same or different and each is —(CR⁶R⁷)ₘ—R⁸, or R⁹ and R¹⁰ together form —(CR⁶R⁷)ₒ—, and

R¹¹ is H or alkyl,

M⁺ is a counterion,

m and n are the same or different and each is 0 or an integer of 1 to 4, and

o is 3 or 4, and

a compound of formula (II):

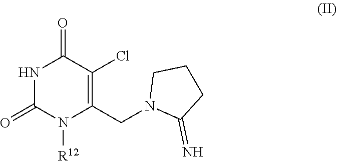

wherein

R¹² is H or —(CR¹³R¹⁴)ₙ—R¹⁵,

each instance of R¹³ and R¹⁴ is the same or different and each is H or alkyl,

R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,

R¹⁶ and R¹⁸ is the same or different and each is H or alkyl,

R¹⁷ is H, alkyl, OH, or SH,

X is O, S, or NR⁵, and

n is 0 or an integer of 1 to 4,

or a pharmaceutically acceptable salt thereof.

In embodiments of these methods, the active agent is 3′-uridylic acid (3′UMP).

In embodiments of these methods, the active agent is 5′-uridylic acid (5′UMP).

In embodiments of these methods, the active agent is citrate. The citrate can be provided in any suitable form, such as citric acid or a salt thereof (e.g., a salt with an alkali metal (Group 1 of the Periodic Table) or an alkaline earth metal (Group 2 of the Periodic Table)).

In embodiments of these methods, the active agent is a tetracycline antibiotic of the formula

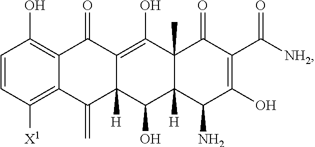

wherein X¹ is H or Cl and salts thereof. When X¹ is H, the tetracycline antibiotic is methacycline (also known as metacycline; and (4S,4aR,5S,5aR,12aR)-4-(dimethylamino)-1,5,10,11,12a-pentahydroxy-6-methylidene-3,12-dioxo-4,4a,5,5a-tetrahydrotetracene-2-carboxamide). In some embodiments, the active agent is methacycline hydrochloride. When X¹ is Cl, the tetracycline antibiotic is meclocycline. In some embodiments, the active agent is meclocycline sulfosalicylate ((4S,4aR,5S,5aR,12aR)-7-chloro-4-(dimethylamino)-1,5,10,11,12a-pentahydroxy-6-methylidene-3,12-dioxo-4,4a,5,5a-tetrahydrotetracene-2-carboxamide; 2-hydroxy-5-sulfobenzoic acid).

In embodiments of these methods, the active agent is an anthracycline antineoplastic agent with a core structure of

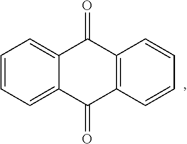 [SMILES: O=C1c2ccccc2C(=O)c2ccccc21]

such as mitoxantrone (1,4-dihydroxy-5,8-bis[2-(2-hydroxyethylamino)ethylamino]anthracene-9,10-dione), epirubicin ((7S,9S)-7-[(2R,4S,5R,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione), including epirubicin hydrochloride, and daunorubicin ((7S,9S)-9-acetyl-7-[(2R,4S,5S,6S)-4-amino-5-hydroxy-6-methyloxan-2-yl]oxy-6,9,11-trihydroxy-4-methoxy-8,10-dihydro-7H-tetracene-5,12-dione), including daunorubicin hydrochloride.

In embodiments of these methods, the active agent is mitoxantrone.

In embodiments of these methods, the active agent is epirubicin hydrochloride.

In embodiments of these methods, the active agent is daunorubicin hydrochloride.

In embodiments of these methods, the active agent is sorafenib (4-[4-({[4-chloro-3-(trifluoromethyl)phenyl]carbamoyl}amino)phenoxy]-N-methylpyridine-2-carboxamide).

In embodiments of these methods, the active agent is sunitinib malate ((2S)-2-hydroxybutanedioic acid; N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide).

In embodiments of these methods, the active agent is primaquine diphosphate (4-N-(6-methoxyquinolin-8-yl)pentane-1,4-diamine diphosphate).

In embodiments of these methods, the active agent is closantel (N-[5-chloro-4-[(4-chlorophenyl)-cyanomethyl]-2-methylphenyl]-2-hydroxy-3,5-diiodobenzamide).

In embodiments of these methods, the active agent is isopropyl ester of N4-hydroxycytidine.

In embodiments of these methods, the active agent is a GpU dinucleotide of formula (I). The compound of formula (I) includes subgenera of formulas (Ia) and (Ib):

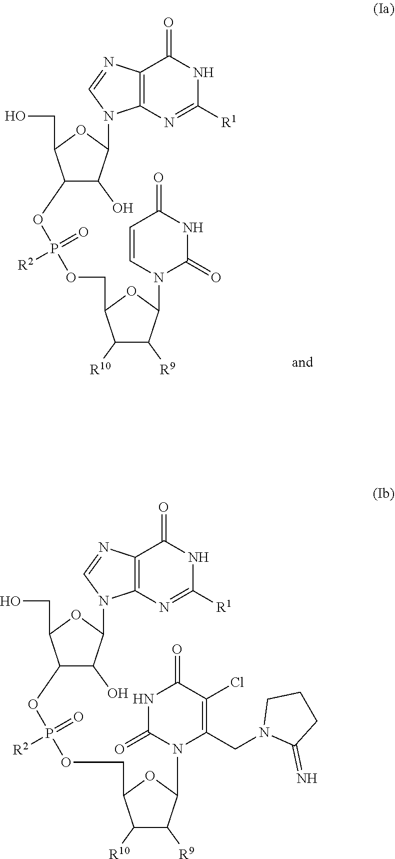

In some embodiments, the compound of formula (I) is formula (Ia), in which R¹ is NH₂, R² is O⁻Na⁺, R⁹ and R¹⁰ are both OH, i.e., GpU dinucleotide.

In other embodiments of formula (I), including formulas (Ia) and (Ib), R¹ is NH₂.

In some embodiments, the compound of formula (I), including formulas (Ia) and (Ib), has R² that is O⁻M⁺. The counterion M⁺ is any suitable cation to balance the charge when R² is O⁻M⁺. For example, M⁺ can be an alkali metal cation (e.g., sodium or potassium), an alkaline earth metal cation (e.g., calcium), or an ammonium cation. Preferably, M⁺ is a cation of lithium, sodium, or potassium.

Other embodiments include the compound of formula (I), including formulas (Ia) and (Ib), in which R² is —(CR⁶R⁷)ₙ—R⁸. The subscript “n” represents the number of CR⁶R⁷ repeat units, in which each substituent, e.g., each R⁶ or R⁷, can be the same or different. The subscript n is 0 or an integer of 1 to 4 (i.e., 1, 2, 3, or 4). Preferably in these embodiments, R⁶ and R⁷ are each H, R⁸ is OH, and n is 2 or 3.

In other embodiments of formula (I), R³ is —CH₂CH₂CH₂CH₂—, —CH₂CH₂CH(CO₂H)CH₂—, or —CH₂CH(CO₂H)CH₂CH₂—.

Other embodiments include compounds of formulas (Ia) and (Ib), in which R⁹ and R¹⁰ are each —(CR⁶R⁷)ₘ—R⁸, in which R⁸ preferably is alkyl or OH. The subscript “m” represents the number of CR⁶R⁷ repeat units, in which each substituent, e.g., each R⁶ or R⁷, can be the same or different. The subscript m is 0 or an integer of 1 to 4 (i.e., 1, 2, 3, or 4), but preferably m is 2, 3, or 4. Preferably, R⁹ is OH, —(CH₂)₂OH, or —(CH₂)₃OH and/or R¹⁰ is OH or alkyl. In other embodiments of formulas (Ia) and (Ib), R⁹ and R¹⁰ together form —(CR⁶R⁷)ₒ—, preferably —CH₂CH₂CH₂—. The subscript “o” represents the number of CR⁶R⁷ repeat units, in which each substituent, e.g., each R⁶ or R⁷, can be the same or different. The subscript o is 3 or 4, but preferably is 3.

In embodiments of these methods, the active agent is a compound of formula (II) or a pharmaceutically acceptable salt thereof. In preferred embodiments of formula (II), R¹² is H, i.e., 5-chloro-6-[(2-imino-1-pyrrolidinyl)methyl]-2,4(1H,3H)-pyrimidinedione (commonly known as tipiracil) or a pharmaceutically acceptable salt thereof, such as tipiracil hydrochloride.

tipiracil is used as a combination drug with trifluridine in the treatment of colorectal cancer and has been approved by FDA. tipiracil inhibits the enzyme thymidine phosphorylase, which metabolizes trifluridine. It may be possible to repurpose tipiracil against a nidovirus that encodes Nsp15 (e.g., SARS CoV-2 virus) with Nsp15 being the drug target.

In other embodiments of formula (II), R¹² is or —(CR¹³R¹⁴)ₙ—R⁵, in which R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸. In these embodiments, X preferably is O, S, or NH, but more preferably X is O. The subscript “n” represents the number of CR¹³R¹⁴ repeat units, in which each substituent, e.g., each R¹³ or R¹⁴, can be the same or different. The subscript n is 0 or an integer of 1 to 4 (i.e., 1, 2, 3, or 4), but preferably n is 2, 3, or 4.

Preferably, R¹⁵ is —OR¹⁶, —NR¹⁶R¹⁷, —C(O)OR¹⁶, —C(O)—NR¹⁶R¹⁷, or —NR¹⁶C(O)OR¹⁸, and n is in integer of 1 to 4 (i.e., 1, 2, 3, or 4). In these embodiments of the compound of formula (I), R¹³ and R¹⁴ preferably are each H, R¹⁶ preferably is H or methyl, R¹⁷ preferably is H, methyl, or OH, R¹⁸ preferably is methyl, and n preferably is an integer of 2 or 3. Exemplary compounds of formula (I) include R¹² that is H, —(CH₂)₂OH, —(CH₂)₃OH, —(CH₂)₂OCH₃, —(CH₂)₃OCH₃, —(CH₂)₂NH₂, —(CH₂)₂N(CH₃)₂, —(CH₂)₂CO₂CH₃, —(CH₂)₃CO₂CH₃, —(CH₂)₄CO₂CH₃, —(CH₂)₂NHCO₂H, or —(CH₂)₂NHCO₂CH₃, or a pharmaceutically acceptable salt thereof.

In any of the embodiments herein, the term “alkyl” implies a straight-chain or branched alkyl substituent containing from, for example, from about 1 to about 8 carbon atoms, e.g., from about 1 to about 6 carbon atoms. Examples of alkyl group include methyl, ethyl, n-propyl, isopropyl, n-butyl, sec-butyl, isobutyl, tert-butyl, n-pentyl, isopentyl, n-hexyl, and the like. This definition also applies wherever “alkyl” occurs as part of a group, such as, e.g., in alkoxy, aminoalkyl, alkylamino, dialkylamino, etc.

Whenever a range of the number of atoms in a structure is indicated (e.g., a C₁₋₈, C₁₋₆, or C₁₋₄ alkyl), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

In any of the embodiments herein, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977). For example, they can be a salt of an alkali metal (e.g., sodium or potassium), alkaline earth metal (e.g., calcium), or ammonium.

In certain embodiments of the compound of formula (II), the compound is a pharmaceutically acceptable salt, preferably a hydrochloride salt.

The compound of formula (II) can include various crystal structures. For example, tipiracil hydrochloride has 3 known crystal forms: Crystal Form I, Crystal Form II, and Crystal Form III. See, e.g., U.S. Pat. Nos. 9,527,833 and 10,457,666, the disclosures of which are incorporated by reference herein in their entireties. Crystal Form I of tipiracil hydrochloride has powder X-ray peaks at two or more angles selected from the group consisting of 11.6°, 17.2°, 17.8°, 23.3°, 27.1°, and 29.3° as a diffraction angle (2θ±0.2°). Crystal Form II of tipiracil hydrochloride has powder X-ray peaks at two or more angles selected from the group consisting of 6.5°, 20.6°, 25.5°, 26.1°, 27.0°, and 30.2° as a diffraction angle (2θ±0.1°). Crystal Form III of tipiracil hydrochloride has powder X-ray peaks at two or more angles selected from the group consisting of 10.5°, 19.6°, 23.7°, 26.2°, and 31.2° as a diffraction angle (2θ±0.1°).

The compound of formula (II) (e.g., tipiracil hydrochloride) preferably is used in the form of Crystal Form I, exhibiting powder X-ray peaks at two or more angles selected from the group consisting of 11.6°, 17.2°, 17.8°, 23.3°, 27.1°, and 29.3° as a diffraction angle (2θ±0.2°).

In preferred aspects of the methods described herein, the compound of formula (II) is tipiracil hydrochloride having a purity of 90% by mass or more (e.g., 92% by mass or more, 95% by mass or more, 97% by mass or more, or 99% by mass or more). More preferably, the tipiracil hydrochloride having a purity of 90% by mass or more has Crystal Form I, as described herein and in U.S. Pat. Nos. 9,527,833 and 10,457,666.

The active agent can be provided in any suitable manner. For example, the active agent can be purchased commercially or synthetically prepared using routine procedures known in the art.

The methods described herein comprise using (e.g., administering) the active agent in the form of a pharmaceutical composition. In particular, a pharmaceutical composition will comprise at least one active agent, as described herein, and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions can be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the pharmaceutical composition is administered orally or intravenously.

In accordance with any of the embodiments, the active agent can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The active agent can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as polyethylene glycol (e.g., PEG400), an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active agent in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The active agent may be made into an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution, such as a mouthwash. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, alcohols, glycols such as glycerin, lipid materials such as fatty acids, mineral oils, phosphoglycerides, collagen, gelatin, and silicone-based materials.

The active agent, alone or in combination with other suitable components, can be made into aerosol formulations to be administered via inhalation. These aerosol formulations can be placed into pressurized acceptable propellants. Suitable propellants include, e.g., a fluorinated hydrocarbon (e.g., trichloromonofluoromethane, dichlorodifluoromethane, chlorodifluoromethane, chlorodifluoroethane, dichlorotetrafluoroethane, heptafluoropropane, tetrafluoroethane, difluoroethane), a hydrocarbon (e.g., propane, butane, isobutane), or a compressed gas (e.g., nitrogen, nitrous oxide, carbon dioxide). They also may be formulated as pharmaceuticals for non-pressured preparations, such as in a nebulizer or an atomizer.

The dose administered to the subject, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular active agent and the desired effect. It will be appreciated by one of skill in the art that various conditions or disease states may require prolonged treatment involving multiple administrations.

The inventive methods comprise using an effective amount of the active agent. An “effective amount” means an amount sufficient to show a meaningful benefit in an individual, cell, or tissue to be treated. A meaningful benefit includes, for example, detectably treating, relieving, or lessening one or more symptoms of a disease caused by a nidovirus that encodes Nsp15 (e.g., inflammation, fluid accumulation), inhibiting, arresting development, preventing, or halting further development of the viral infection or disease, reducing the incidence of a disease caused by nidovirus that encodes Nsp15, preventing a disease caused by nidovirus that encodes Nsp15 from occurring in a subject, cell, or tissue at risk thereof but yet to be diagnosed, and/or detectably inhibit one or more active sites of Nsp15 in a subject, cell, or tissue. The meaningful benefit observed in the subject, cell, or tissue to be treated can be to any suitable degree (10, 20, 30, 40, 50, 60, 70, 80, 90% or more). In some aspects, one or more symptoms of the disease are prevented, reduced, halted, or eliminated subsequent to administration of an active agent described herein, thereby effectively treating the disease to at least some degree.

Effective amounts may vary depending upon the biological effect desired in the individual, cell and/or tissue to be treated, condition to be treated, and/or the specific characteristics of the active agent. In this respect, any suitable dose of the active agent can be administered to the subject (e.g., human), cell, or tissue. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the active agent desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

In any of the embodiments of the methods described herein, the nidovirus that encodes Nsp15 can be a coronavirus, an arterivirus, or a torovirus. In certain embodiments, the nidovirus is a coronavirus, such as SARS-CoV, SARS-CoV-2, or MERS-CoV. Preferably, the coronavirus is SARS-CoV-2. In other embodiments, the nidovirus is an arterivirus. In other embodiments, the nidovirus is a torovirus (e.g., renitovirus).

The disease caused by the nidovirus can be, for example, coronavirus disease (COVID-19), severe acute respiratory syndrome (SARS) virus, Middle East respiratory syndrome (MERS), a respiratory disease (e.g., pneumonia, bronchitis, and pleural effusion), an inflammatory disease (e.g., inflammation, COVID-19-induced inflammation, pediatric multi-system inflammatory syndrome (PMIS)), reproductive and respiratory syndrome virus (PRRSV), equine arteritis virus (EAV), or gastroenteritis.

Without wishing to be bound by any theory, it is believed that NEndoU activity interferes with the innate immune response, facilitating evasion of host pattern recognition receptor MDA5. EndoU activity is considered to be essential in nidovirus (e.g., coronavirus) biology. It was shown to degrade viral RNA the polyuridine extensions on negative-sense strand of RNA. Nsp15 is highly conserved in nidoviruses, such as coronavirus, suggesting that its activity is important for virus replication. It is considered that inhibitors of Nsp15 of a nidovirus can effectively prevent or reduce viral replication, thereby treating a disease caused by the nidovirus. Thus, in accordance with the methods described herein, an Nsp15 endoribonuclease of the nidovirus is inhibited by the active agent.

The method of inhibiting Nsp15 endoribonuclease can be an in vivo treatment in a subject in need thereof, as described herein, or an in vitro or ex vivo treatment of a cell and/or tissue. The cell can be from any suitable tissue, such as tissue of the lung.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

A subject in need thereof is any one that has come in contact with, suspected to have come in contact with, or expected to come into contact with a nidovirus that encodes Nsp15 (e.g., SARS-CoV-2). At risk subjects for developing a disease caused by a nidovirus that encodes Nsp15 include, for example, people aged 40 and older (particularly people aged 60 and older), people with one more underlying conditions (e.g., cardiovascular disease, diabetes, chronic respiratory disease, asthma, liver disease, chronic kidney disease undergoing dialysis, high blood pressure, obesity (e.g., a body mass index (BMI) of 30 or higher, especially 40 or higher), and cancer), people that are immunocompromised (e.g., due to a condition such as smoking, cancer treatment, bone marrow or organ transplantation, HIV, AIDs, and prolonged use of corticosteroids and other immune weakening treatments), and people living in a nursing home or a long-term care facility.

In accordance with methods described herein, the invention provides novel compounds of formula (I), which are defined as a compound of formula (I′):

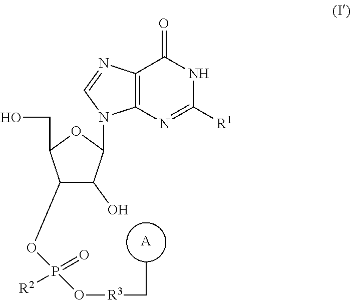

wherein

ring A is

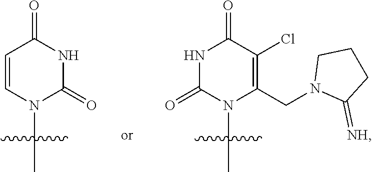 [SMILES: *n1ccc(=O)[nH]c1=O ; *n1c(CN2CCCC2=N)c(Cl)c(=O)[nH]c1=O]

R¹ is —NR⁴R⁵,

R² is O⁻M⁺ or —(CR⁶R⁷)ₙ—R⁸,

R³ is 1′,5′-ribosyl of the formula

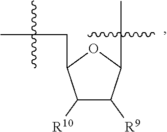

—CH₂CH₂CH₂CH₂—, —CH₂CH₂CH(CO₂H)CH₂—, or —CH₂CH(CO₂H)CH₂CH₂—,

R⁴ and R⁵ is the same or different and each is H or alkyl,

each instance of R⁶ and R⁷ is the same or different and each is H or alkyl,

R⁸ is alkyl, —OR¹¹, —SR¹¹, or —NR⁴R⁵, and

R⁹ and R¹⁰ are the same or different and each is —(CR⁶R⁷)ₘ—R⁸, or R⁹ and R¹⁰ together form —(CR⁶R⁷)ₒ—, and

R¹¹ is H or alkyl,

M⁺ is a counterion,

m and n are the same or different and each is 0 or an integer of 1 to 4, and

o is 3 or 4,

provided that the compound is not GpU dinucleotide.

The compound of formula (I′) includes subgenera of formulas (Ia′) and (Ib′):

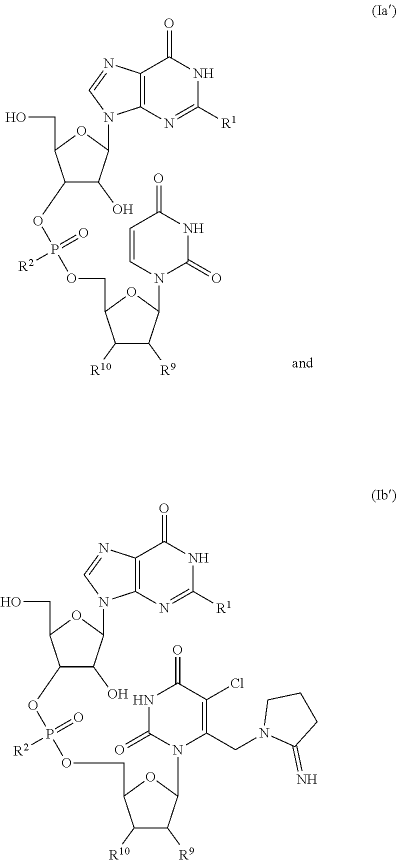

In embodiments of formula (I′), including formulas (Ia′) and (Ib′), R¹ is —NR⁴R⁵, and R⁴ and R⁵ is the same or different and each is H or alkyl, provided that the compound of formula (I′) is not GpU dinucleotide.

In any of these embodiments, the compound of formula (I′), including formulas (Ia′) and (Ib′), has R² that is O⁻M⁺, provided that the compound of formula (I′) is not GpU dinucleotide. The counterion M⁺ is any suitable cation to balance the charge when R² is O⁻M⁺. For example, M⁺ can be an alkali metal cation (e.g., sodium or potassium), an alkaline earth metal cation (e.g., calcium), or an ammonium cation. Preferably, M⁺ is a cation of lithium, sodium, or potassium.

In any of these embodiments, the compound of formula (I′), including formulas (Ia′) and (Ib′), has R² that is —(CR⁶R⁷)ₙ—R⁸. The subscript “n” represents the number of CR⁶R⁷ repeat units, in which each substituent, e.g., each R⁶ or R⁷, can be the same or different. The subscript n is 0 or an integer of 1 to 4 (i.e., 1, 2, 3, or 4). Preferably in these embodiments, R⁶ and R⁷ are each H, R⁸ is OH, and n is 2 or 3.

In any of these embodiments of formula (I′), R³ is —CH₂CH₂CH₂CH₂—, —CH₂CH₂CH(CO₂H)CH₂—, or —CH₂CH(CO₂H)CH₂CH₂—.

In any of these embodiments, compounds of formulas (Ia′) and (Ib′), include R⁹ and R¹⁰ that are each —(CR⁶R⁷)ₘ—R⁸, in which R⁸ preferably is alkyl or OH, provided that the compound of formula (I′) is not GpU dinucleotide. The subscript “m” represents the number of CR⁶R⁷ repeat units, in which each substituent, e.g., each R⁶ or R⁷, can be the same or different. The subscript m is 0 or an integer of 1 to 4 (i.e., 1, 2, 3, or 4), but preferably m is 2, 3, or 4. Preferably, R⁹ is OH, —(CH₂)₂OH, or —(CH₂)₃OH and/or R¹⁰ is OH or alkyl. In any of these embodiments of formulas (Ia′) and (Ib′), R⁹ and R¹⁰ together form —(CR⁶R⁷)ₒ—, preferably —CH₂CH₂CH₂—. The subscript “o” represents the number of CR⁶R⁷ repeat units, in which each substituent, e.g., each R⁶ or R⁷, can be the same or different. The subscript o is 3 or 4, but preferably is 3.

The invention further provides novel compounds of formula (II), which are defined as a compound of formula (II′):

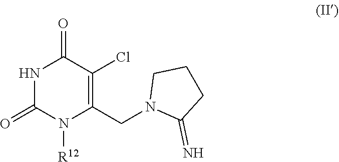

wherein

R¹² is —(CR¹³R¹⁴)ₙ—R¹⁵,

each instance of R¹³ and R¹⁴ is the same or different and each is H or alkyl,

R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,

R¹⁶ and R¹⁸ is the same or different and each is H or alkyl,

R¹⁷ is H, alkyl, OH, or SH,

X is O, S, or NR⁵, and

n is 0 or an integer of 1 to 4,

or a pharmaceutically acceptable salt thereof.

In some preferred compounds of formula (II′): (i) R¹³ and R¹⁴ are each H and/or (ii) R¹⁵ is —OR¹⁶, —NR¹⁶R¹⁷, —C(O)OR¹⁶, —C(O)—NR¹⁶R¹⁷, or —NR¹⁶C(O)OR¹⁸, (iii) n is in integer of 1 to 4, and/or (iv) R¹⁶ is H or methyl, R¹⁷ is H, methyl, or OH, and R¹⁸ is methyl. Specific examples of the compound of formula (I′) include compounds in which R¹² is —(CH₂)₂OH, —(CH₂)₃OH, —(CH₂)₂OCH₃, —(CH₂)₃OCH₃, —(CH₂)₂NH₂, —(CH₂)₂N(CH₃)₂, —(CH₂)₂CO₂CH₃, —(CH₂)₃CO₂CH₃, —(CH₂)₄CO₂CH₃, —(CH₂)₂NHCO₂H, —(CH₂)₂NHCO₂CH₃, and pharmaceutically acceptable salts thereof. In some preferred embodiments, the compounds of formula (II′) are in the form of a hydrochloride salt.

The compound of formula (II′), as described herein, can be prepared using any suitable synthesis for forming a tertiary amine. For example, commercially purchased tipiracil can be reacted with R¹⁵(CR¹³R¹⁴)ₙ-L, in which R¹³⁻¹⁵ are as described herein and L is a suitable leaving group (e.g., such as halo (I⁻, Br⁻, or Cl⁻), sulfate, sulfonate, etc.) to provide the N-substituted derivative of formula (II′). Preferably, the reaction takes place in the presence of a base (e.g., KOH, NaOH, triethylamine, etc.).

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The invention is further illustrated by the following aspects.

Aspect 1. A method of treating a disease caused by a nidovirus that encodes Nsp15 in a subject in need thereof comprising administering to the subject a therapeutically effective amount of an active agent selected from 3′-uridylic acid, 5′-uridylic acid, citrate, a tetracycline antibiotic of the formula

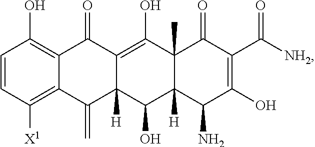

wherein X¹ is H or Cl and salts thereof,
an anthracycline antineoplastic agent with a core structure of

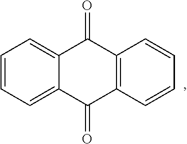 [SMILES: O=C1c2ccccc2C(=O)c2ccccc21]

sorafenib, sunitinib malate, primaquine diphosphate, closantel, an isopropyl ester of N4-hydroxycytidine,
a GpU dinucleotide of formula (I):

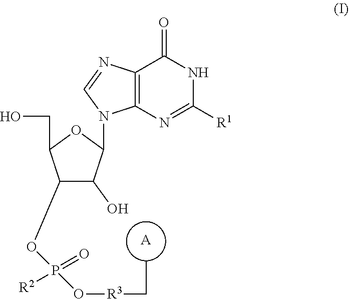

wherein

ring A is

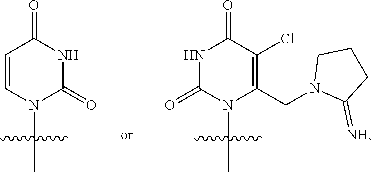 [SMILES: *n1ccc(=O)[nH]c1=O ; *n1c(CN2CCCC2=N)c(Cl)c(=O)[nH]c1=O]

R¹ is —NR⁴R⁵,

R² is O⁻M⁺ or —(CR⁶R⁷)ₙ—R⁸,

R³ is 1′,5′-ribosyl of the formula

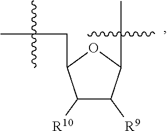

—CH₂CH₂CH₂CH₂—, —CH₂CH₂CH(CO₂H)CH₂—, or —CH₂CH(CO₂H)CH₂CH₂—,

R⁴ and R⁵ is the same or different and each is H or alkyl,

each instance of R⁶ and R⁷ is the same or different and each is H or alkyl, R⁸ is alkyl, —OR¹¹, —SR¹¹, or —NR⁴R⁵, and

R⁹ and R¹⁰ are the same or different and each is —(CR⁶R⁷)ₘ—R⁸, or R⁹ and R¹⁰ together form —(CR⁶R⁷)ₒ—, and

R¹¹ is H or alkyl,

M⁺ is a counterion,

m and n are the same or different and each is 0 or an integer of 1 to 4, and

o is 3 or 4, and
a compound of formula (II):

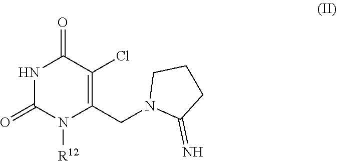

wherein
R¹² is H or —(CR¹³R¹⁴)ₙ—R⁵,

each instance of R¹³ and R¹⁴ is the same or different and each is H or alkyl,

R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,

R¹⁶ and R¹⁸ is the same or different and each is H or alkyl,

R¹⁷ is H, alkyl, OH, or SH,

X is O, S, or NR⁵, and

n is 0 or an integer of 1 to 4,

or a pharmaceutically acceptable salt thereof.

Aspect 2. A method of inhibiting an Nsp15 endoribonuclease of a nidovirus that encodes Nsp15 comprising contacting the Nsp15 endoribonuclease with an effective amount of an active agent selected from 3′-uridylic acid, 5′-uridylic acid, citrate, a tetracycline antibiotic of the formula

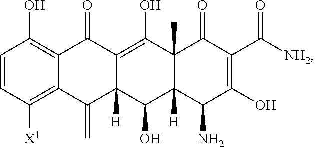

wherein X¹ is H or Cl and salts thereof,
an anthracycline antineoplastic agent with a core structure of

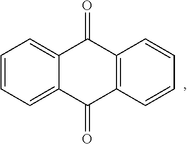 [SMILES: O=C1c2ccccc2C(=O)c2ccccc21]

sorafenib, sunitinib malate, primaquine diphosphate, closantel, an isopropyl ester of N4-hydroxycytidine, a GpU dinucleotide of formula (I):

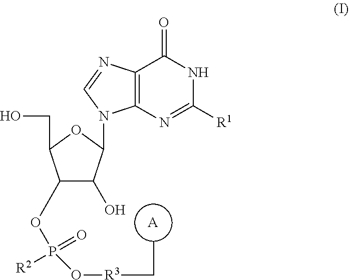

wherein

ring A is

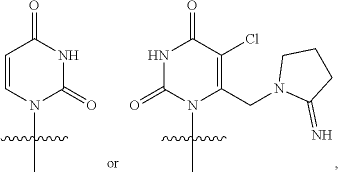

R¹ is —NR⁴R⁵,

R² is O⁻M⁺ or —(CR⁶R⁷)ₙ—R⁸,

R³ is 1′,5′-ribosyl of the formula

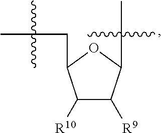

—CH₂CH₂CH₂CH₂—, —CH₂CH₂CH(CO₂H)CH₂—, or —CH₂CH(CO₂H)CH₂CH₂—,

R⁴ and R⁵ is the same or different and each is H or alkyl,

each instance of R⁶ and R⁷ is the same or different and each is H or alkyl, R⁸ is alkyl, —OR¹¹, —SR¹¹, or —NR⁴R⁵, and

R⁹ and R¹⁰ are the same or different and each is —(CR⁶R⁷)ₘ—R⁸, or R⁹ and R¹⁰ together form —(CR⁶R⁷)ₒ—, and

R¹¹ is H or alkyl,

M⁺ is a counterion,

m and n are the same or different and each is 0 or an integer of 1 to 4, and

o is 3 or 4, and
a compound of formula (II):

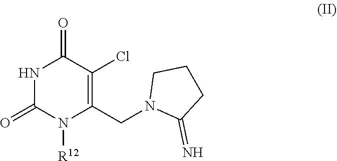

wherein
R¹² is H or —(CR¹³R¹⁴)ₙ—R¹⁵,

each instance of R¹³ and R¹⁴ is the same or different and each is H or alkyl,

R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,

R¹⁶ and R¹⁸ is the same or different and each is H or alkyl,

R¹⁷ is H, alkyl, OH, or SH,

X is O, S, or NR⁵, and

n is O or an integer of 1 to 4,

or a pharmaceutically acceptable salt thereof.

Aspect 3. The method of aspect 1 or 2, wherein the active agent is 3′-uridylic acid.

Aspect 4. The method of aspect 1 or 2, wherein the active agent is 5′-uridylic acid.

Aspect 5. The method of aspect 1 or 2, wherein the active agent is citrate.

Aspect 6. The method of aspect 1 or 2, wherein the active agent is a tetracycline antibiotic of the formula

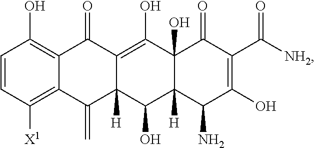

wherein X¹ is H or Cl and salts thereof.

Aspect 7. The method of aspect 1 or 2, wherein the active agent is an anthracycline antineoplastic agent with a core structure of

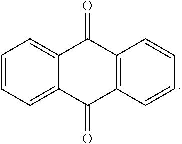 [SMILES: O=C1c2ccccc2C(=O)c2ccccc21]

Aspect 8. The method of aspect 1 or 2, wherein the active agent is sorafenib.

Aspect 9. The method of aspect 1 or 2, wherein the active agent is sunitinib malate.

Aspect 10. The method of aspect 1 or 2, wherein the active agent is primaquine diphosphate.

Aspect 11. The method of aspect 1 or 2, wherein the active agent is closantel.

Aspect 12. The method of aspect 1 or 2, wherein the active agent is an isopropyl ester of N4-hydroxycytidine.

Aspect 13. The method of aspect 1 or 2, wherein the active agent is a GpU dinucleotide of formula (I).

Aspect 14. The method of aspect 13, wherein ring A is

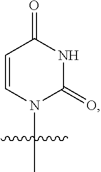 [SMILES: *n1ccc(=O)[nH]c1=O]

R¹ is NH₂, R² is O⁻Na⁺, R³ is 1′,5′-ribosyl of the formula

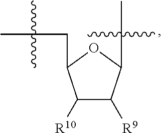

and R⁹ and R¹⁰ are both OH.

Aspect 15. The method of aspect 1 or 2, wherein the active agent a compound of formula (II) or a pharmaceutically acceptable salt thereof.

Aspect 16. The method of aspect 15, wherein R¹² is H or a pharmaceutically acceptable salt thereof.

Aspect 17. The method of aspect 15, wherein R¹² is or —(CR¹³R¹⁴)ₙ—R¹⁵, R¹⁵ is —OR¹⁶, —NR¹⁶R¹⁷, —C(O)OR¹⁶, —C(O)—NR¹⁶R¹⁷, or —NR¹⁶C(O)OR¹⁸, and n is in integer of 1 to 4, or a pharmaceutically acceptable salt thereof.

Aspect 18. The method of aspect 17, wherein R¹³ and R¹⁴ are each H, R¹⁶ is H or methyl, R¹⁷ is H, methyl, or OH, R¹⁸ is methyl, and n is an integer of 2 or 3, or a pharmaceutically acceptable salt thereof.

Aspect 19. The method of aspect 15, wherein R¹² is —(CH₂)₂OH, —(CH₂)₃OH, —(CH₂)₂OCH₃, —(CH₂)₃OCH₃, —(CH₂)₂NH₂, —(CH₂)₂N(CH₃)₂, —(CH₂)₂CO₂CH₃, —(CH₂)₃CO₂CH₃, —(CH₂)₄CO₂CH₃, —(CH₂)₂NHCO₂H, or —(CH₂)₂NHCO₂CH₃, or a pharmaceutically acceptable salt thereof.

Aspect 20. The method of any one of aspects 15-19, wherein the compound of formula (II) is in the form of a hydrochloride salt.

Aspect 21. The method of aspect 16 or 20, wherein the compound of formula (II) is Crystal Form I, exhibiting powder X-ray peaks at two or more angles selected from the group consisting of 11.6°, 17.2°, 17.8°, 23.3°, 27.1°, and 29.3° as a diffraction angle (2θ±0.2°).

Aspect 22. The method of aspect 21, wherein the compound of formula (II), wherein the compound of formula (I) is 5-chloro-6-(2-iminopyrrolidin-1-yl)methyl-2,4(1H,3H)-pyrimidinedione hydrochloride having a purity of at least 90% by mass.

Aspect 23. The method of any one of aspects 1-22, wherein the nidovirus is a coronavirus.

Aspect 24. The method of aspect 23, wherein the coronavirus is SARS-CoV-2.

Aspect 25. The method of any one of aspects 1-22, wherein the nidovirus is an arterivirus.

Aspect 26. The method of any one of aspects 1-22, wherein the nidovirus is a torovirus.

Aspect 27. The method of any one of aspects 1 and 3-26, wherein the disease is coronavirus disease (COVID-19), severe acute respiratory syndrome (SARS) virus, Middle East respiratory syndrome (MERS), a respiratory disease, an inflammatory disease, reproductive and respiratory syndrome virus (PRRSV), equine arteritis virus (EAV), or gastroenteritis.

Aspect 28. The method of any one of aspects 1 and 3-27, wherein an Nsp15 endoribonuclease of the nidovirus is inhibited by the active agent.

Aspect 29. A compound of formula (I′):

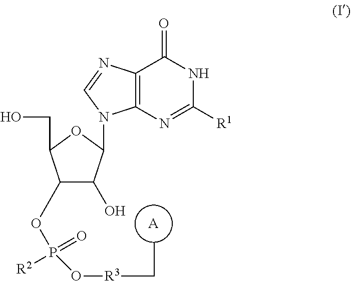

wherein

ring A is

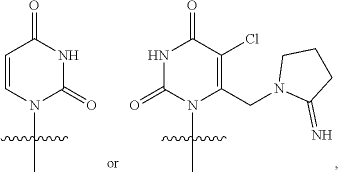

R¹ is —NR⁴R⁵,

R² is O⁻M⁺ or —(CR⁶R⁷)ₙ—R⁸,

R³ is 1′,5′-ribosyl of the formula

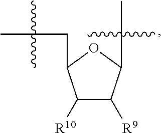

—CH₂CH₂CH₂CH₂—, —CH₂CH₂CH(CO₂H)CH₂—, or —CH₂CH(CO₂H)CH₂CH₂—,

R⁴ and R⁵ is the same or different and each is H or alkyl,

each instance of R⁶ and R⁷ is the same or different and each is H or alkyl, R⁸ is alkyl, —OR¹¹, —SR¹¹, or —NR⁴R⁵, and

R⁹ and R¹⁰ are the same or different and each is —(CR⁶R⁷)ₘ—R⁸, or R⁹ and R¹⁰ together form —(CR⁶R⁷)ₒ—, and

R¹¹ is H or alkyl,

M⁺ is a counterion,

m and n are the same or different and each is 0 or an integer of 1 to 4, and

o is 3 or 4,
provided that the compound of formula (I′) is not GpU dinucleotide.

Aspect 30. A compound of formula (II′):

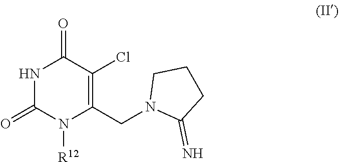

wherein

R¹² is —(CR¹³R¹⁴)ₙ—R¹⁵,

each instance of R¹³ and R¹⁴ is the same or different and each is H or alkyl,

R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,

R¹⁶ and R¹⁸ is the same or different and each is H or alkyl,

R¹⁷ is H, alkyl, OH, or SH,

X is O, S, or NR⁵, and

n is 0 or an integer of 1 to 4,

or a pharmaceutically acceptable salt thereof.

Aspect 31. The compound of aspect 30, wherein R¹³ and R¹⁴ are each H, or a pharmaceutically acceptable salt thereof.

Aspect 32. The compound of aspect 30 or 31, wherein R¹⁵ is —OR¹⁶, —NR¹⁶R¹⁷, —C(O)OR¹⁶, —C(O)—NR¹⁶R¹⁷, or —NR¹⁶C(O)OR¹⁸, and n is in integer of 1 to 4, or a pharmaceutically acceptable salt thereof.

Aspect 33. The compound of aspect 32, wherein R¹⁶ is H or methyl, R¹⁷ is H, methyl, or OH, R¹⁸ is methyl, and n is an integer of 2 or 3, or a pharmaceutically acceptable salt thereof.

Aspect 34. The compound of aspect 30, wherein R¹² is —(CH₂)₂OH, —(CH₂)₃OH, —(CH₂)₂OCH₃, —(CH₂)₃OCH₃, —(CH₂)₂NH₂, —(CH₂)₂N(CH₃)₂, —(CH₂)₂CO₂CH₃, —(CH₂)₃CO₂CH₃, —(CH₂)₄CO₂CH₃, —(CH₂)₂NHCO₂H, or —(CH₂)₂NHCO₂CH₃, or a pharmaceutically acceptable salt thereof.

The following examples further illustrate the invention but, of course, should not be construed as in any way limiting its scope.

### EXAMPLES

The following materials and methods were used.

3′-CMP was obtained from Sigma. Crude [gamma-³²P]ATP was purchased from PerkinElmer (Waltham, Mass.). T4 Polynucleotide Kinase (3′ phosphatase minus) was from New England BioLabs (Ipswich, Mass.). All other chemicals were reagent grade.

[5′-³²P]pCp was prepared by phosphorylation of 3′-CMP as described by England et al. (Methods Enzymol, 65, 65-74 (1980)).

RNA eicosamers were synthetized by runoff transcription of synthetic double-stranded DNA templates published by Sherlin et al. (RNA, 7, 1671-1678 (2001)). 5′-³²P-labeled RNA eicosamers and 3′-³²P-labeled RNA octamers were prepared according to Zwieb et al. (Biochemistry, 40, 9587-9595 (2001)) and England et al. (Methods Enzymol, 65, 65-74 (1980)), respectively.

Protein was expressed and purified using protocol developed by Kim et al. (Protein science: a publication of the Protein Society, doi:10.1002/pro.3873 (2020)). Briefly, a 41 culture of LB Lennox medium was grown at 37° C. (190 rpm) in the presence of ampicillin 150 mg/ml. Once the culture reached OD₆₀₀˜1.0, the temperature setting was changed to 4° C. When the bacterial suspension cooled down to 18° C., it was supplemented with the following components to indicated concentration: 0.2 mM isopropyl β-D-1-thiogalactopyranoside (IPTG), 0.1% glucose, and 40 mM K₂HPO₄. The temperature was set to 18° C. for 20 h incubation. Bacterial cells were harvested by centrifugation at 7,000 g and cell pellets were resuspended in a 12.5 ml lysis buffer (500 mM NaCl, 5% (v/v) glycerol, 50 mM 4-(2-hydroxyethyl)-1-piperazineethanesulfonic acid (HEPES) pH 8.0, 20 mM imidazole, and 1 mM tris(2-carboxyethyl)phosphine (TCEP)) per liter culture and sonicated at 120 W for 5 minutes (4 sec ON, 20 sec OFF). The cellular debris was removed by centrifugation at 30,000 g for 1 h at 4° C. Supernatant was mixed with 4 ml of Ni²⁺ SEPHAROSE™ (GE Healthcare Life Sciences, Chicago, Ill.) equilibrated with lysis buffer supplemented to 50 mM imidazole pH 8.0 and suspension was applied on a KIMBLE™ FLEX-COLUMN™ (420400-2510) (DWK Life Sciences, Milville, N.J.) connected to VAC-MAN™ vacuum manifold (Promega, Madison, Wis.). Unbound proteins were washed out via controlled suction with 160 ml of lysis buffer (50 mM imidazole). Bound proteins were eluted with 20 ml of lysis buffer supplemented imidazole to 500 mM pH 8.0. Then 1 mM TCEP was added followed by Tobacco Etch Virus (TEV) protease treatment at a 1:20 protease:protein ratio. The solution was left at 4° C. overnight. For this particular construct, TEV protease was not able to cleave off the His tag. Nsp15 was successfully separated from TEV protease on SUPERDEX™ 200 column (Cytiva, Marlborough, Mass.), equilibrated in lysis buffer where 10 mM β-mercaptoethanol was replaced by 1 mM TCEP. Fractions containing Nsp15 were collected. Lysis buffer was replaced on 30 kDa MWCO filter (molecular weight cut off filter) (Amicon-Millipore, Burlington, Mass.) via 10× concentration/dilution repeated 3 times to crystallization buffer (150 mM NaCl, 20 mM HEPES pH 7.5, 1 mM TCEP). Final concentration of Nsp15 was 19 mg/ml.

The synthesis of 2′,3′-cyclic uridine vanadate was modified from syntheses set forth in Borah et al. (Biochemistry 24, 2058-2067 (1985)) and Ladner et al. (Acta Crystallogr D Biol Crystallogr 53, 290-301 (1997)) to yield higher concentration of uridine vanadate. In particular, 1 mg of ammonium meta vanadate was dissolved in 200 μl of 60° C. water and 2.93 mg of uridine was added then incubated for 10 min at the same temperature to obtain ˜40 mM uridine vanadate solution. Without further purification, this solution and protein were mixed in a 10:1 molar ratio to set up crystals.

### Example 1

This example demonstrates binding of molecules to the active site of Nsp15.

X-ray crystallographic studies were performed on non-structural protein 15 (Nsp15) in the presence of various molecules. Table 1 shows the molecule and the identification number of the determined structure deposited with the Protein Data Bank (PDB).

TABLE 1

| Molecule | PDB ID No. |
|---|---|
| tipiracil (5-chloro-6-(2- | 6WXC |
| iminopyrrolidin-1-yl)methyl-2,4(1H,3H)- | |
| pyrimidinedione, DB09343, Pub Chem 6323266) | |
| 5′GpU RNA Dinucleotide (5′-3′) | 6X1B |
| 5′UMP (substrate derivative) | 6WLC |
| 3′UMP | 6X4I |
| Citrate | 6W01 |
| uridine 2′,3′-vanadate (UV) | 7KIL |

SARS-CoV-2 Nsp15 protein was crystallized with 5′UMP, 3′UMP, 5′GpU, uridine 2′,3′-vanadate (UV) and tipiracil using methods described previously (Kim et al., Protein science: a publication of the Protein Society, doi:10.1002/pro.3873 (2020)). In particular, all complexes, except for Nsp15/UV, Nsp15/5′UMP, Nsp15/3′UMP, Nsp15/5′GpU, and Nsp15/tipiracil were prepared by mixing 5-15 mM of each ligand with 0.2 mM Nsp15 and incubated for at least 30 minutes before crystallization. Crystals of Nsp15/3′UMP, Nsp15/5′GpU, and Nsp15/tipiracil, the hexagonal crystal form, in P6₃ space group, were obtained from MCSG1 screen A3 condition containing 0.2 M sodium chloride, 0.1 M sodium/potassium phosphate pH 6.2, and 10% (w/v) PEG8000 (Anatrace, Maumee, Ohio). These crystals diffracted x-rays to 1.85, 1.93 and 1.85 Å for Nsp15/3′UMP, Nsp15/5′GpU, and Nsp15/tipiracil, respectively. The crystals of Nsp15/5′UMP and Nsp15/uridine vanadate complexes grew from the Crystal Screen Classic HTP (Jena Bioscience, Germany) C2 condition containing 16% (w/v) PEG4000, 0.1 M Tris (tris(hydroxymethyl)aminomethane) pH 8.5, 0.2 M sodium acetate and diffracted to 1.82 Å and 2.25 Å, respectively. To achieve higher occupancies of the ligands in the structures, except for uridine vanadate, each ligand compound was added to the cryo-solution and the co-crystal was soaked for 2-3 min before frozen in the liquid nitrogen.

The x-ray diffraction experiments were carried out at the Structural Biology Center 19-ID beamline at the Advanced Photon Source, Argonne National Laboratory. The diffraction images were recorded at 100 K at the wavelength of 0.9793 Å from all crystal forms on the PILATUS3×6M detector (Dectris, Baden, Switzerland) using 0.5° rotation and 0.5 sec exposure for 140°, 180°, 110°, 2100 and 125° for Nsp15/5′UMP, Nsp15/3′UMP, Nsp15/5′GpU, Nsp15/tipiracil and Nsp15/UV, respectively. The data were integrated and scaled with the HKL3000 suite (HKL Research, Inc., Charlottesville, Va.) (Minor et al., Acta Crystallographica Section D-Biological Crystallography 62, 859-866 (2006)). Intensities were converted to structure factor amplitudes in the Ctruncate program (French et al., Acta Crystallographica Section A 34, 517-525, (1978); and Padilla et al., Acta Crystallographica Section D-Biological Crystallography 59, 1124-1130 (2003)) from the CCP4 package (Collaborative Computational Project, number 4) (Winn et al., Acta Crystallographica Section D-Biological Crystallography 67, 235-242 (2011)) and using the apo-form SARS-CoV-2 Nsp15 structure (PDB id: 6VWW) as a search model, the structures were determined using Molrep (Vagin et al., Acta Crystallographica Section D-Biological Crystallography 66, 22-25 (2010)), all implemented in the HKL3000 software package. The initial solutions were refined, both rigid-body refinement and regular restrained refinement by REFMAC (Winn et al., Acta Crystallographica Section D-Biological Crystallography 67, 235-242 (2011); and Murshudov et al., Acta Crystallographica Section D-Biological Crystallography 53, 240-255 (1997)) as a part of HKL3000. The models including the ligands were manually adjusted using COOT (Enmsley et al., Acta Crystallographica Section D-Biological Crystallography 60, 2126-2132 (2004)) and then iteratively refined using COOT and PHENIX (Phython-based Hierarchical Environment for Integrated Xtallography) (Adams et al., Acta Crystallographica Section D-Biological Crystallography 66, 213-221 (2010)). Throughout the refinement, the same 5% of reflections were kept out throughout from the refinement (in both REFMAC and PHENIX refinement). The stereochemistry of the structure was checked with PROCHECK (Laskowski et al., Journal of Applied Crystallography 26, 283-291 (1993)) and the Ramachandran plot and validated with the PDB validation server. For the structures Nsp15/5′UMP, Nsp15/3′UMP, Nsp15/5′GpU, Nsp15/tipiracil, and Nsp15/UV, the Ramachandran favored/allowed/outliers were 98.12/1.88/0.0, 97.38/2.47/0.15, 97.39/2.61/0.0, 97.97/2.03/0.0, and 96.96/3.04/0.0%, respectively.

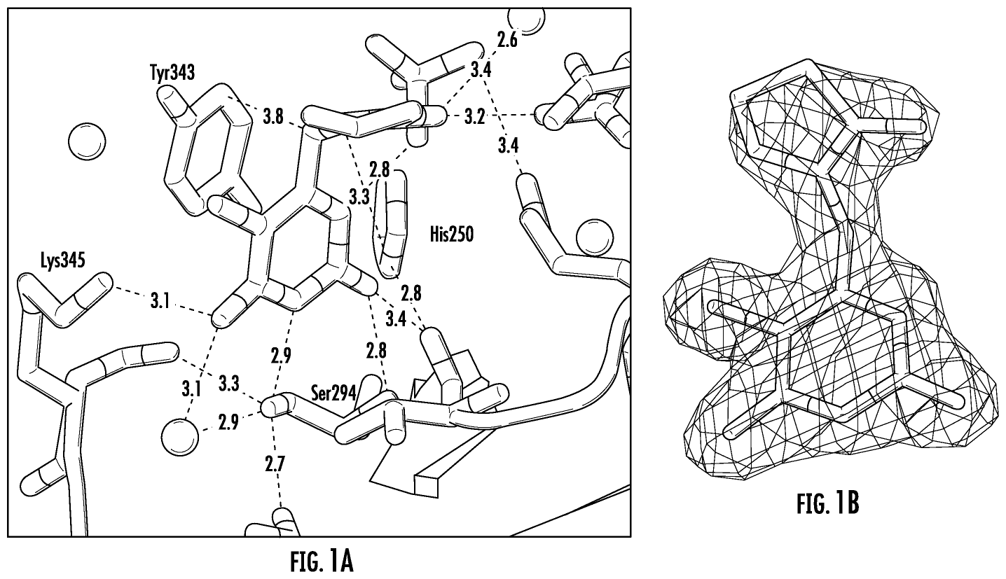

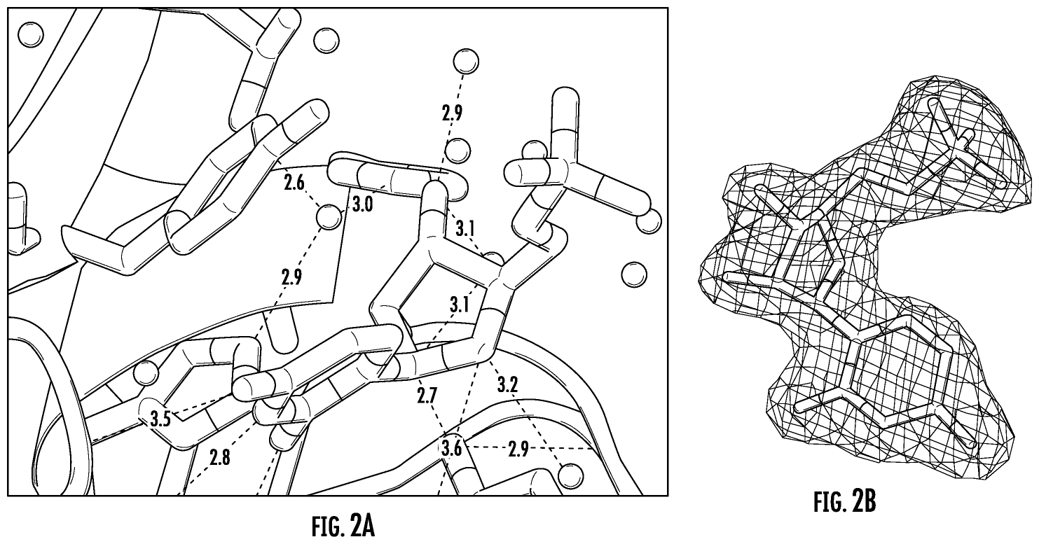

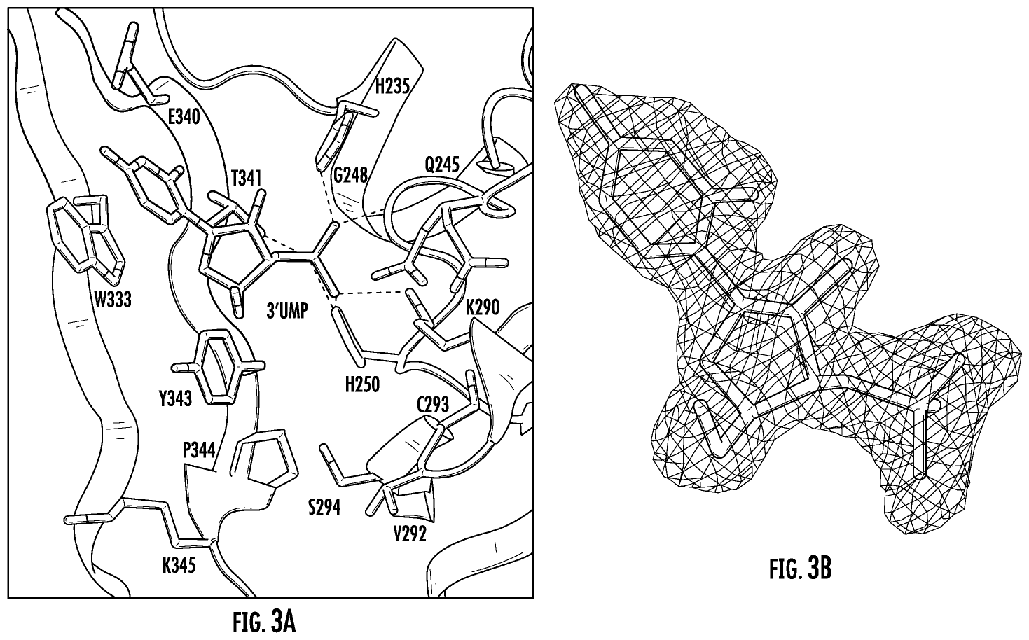

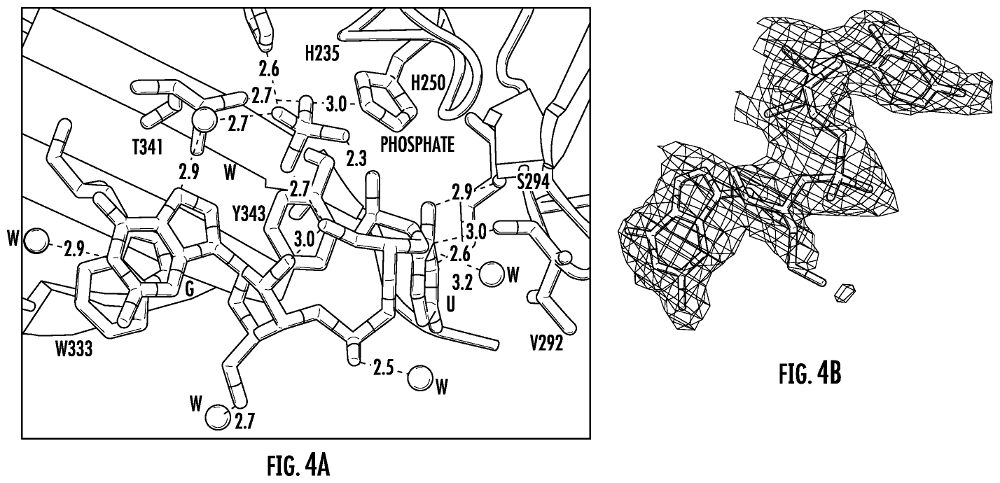

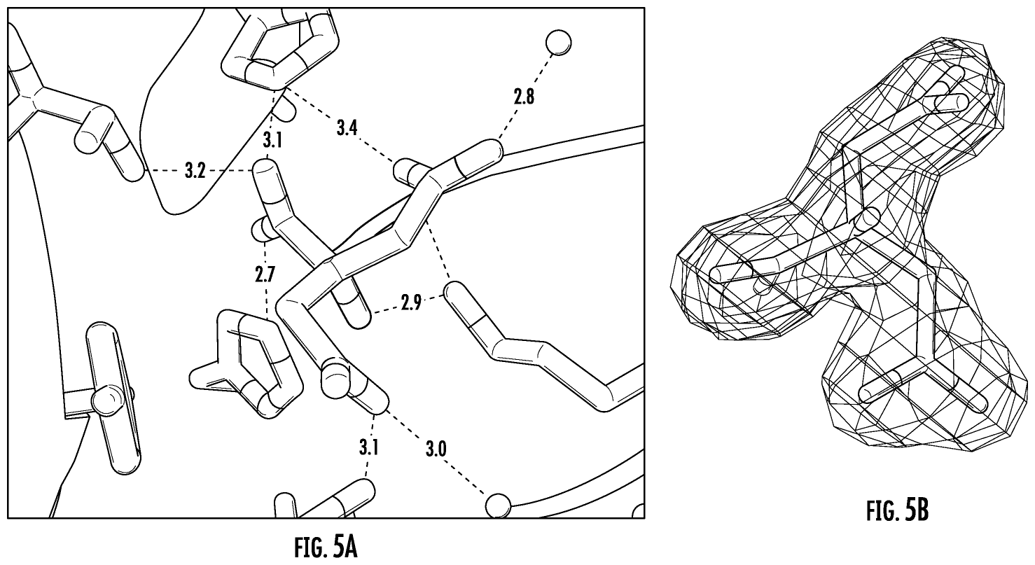

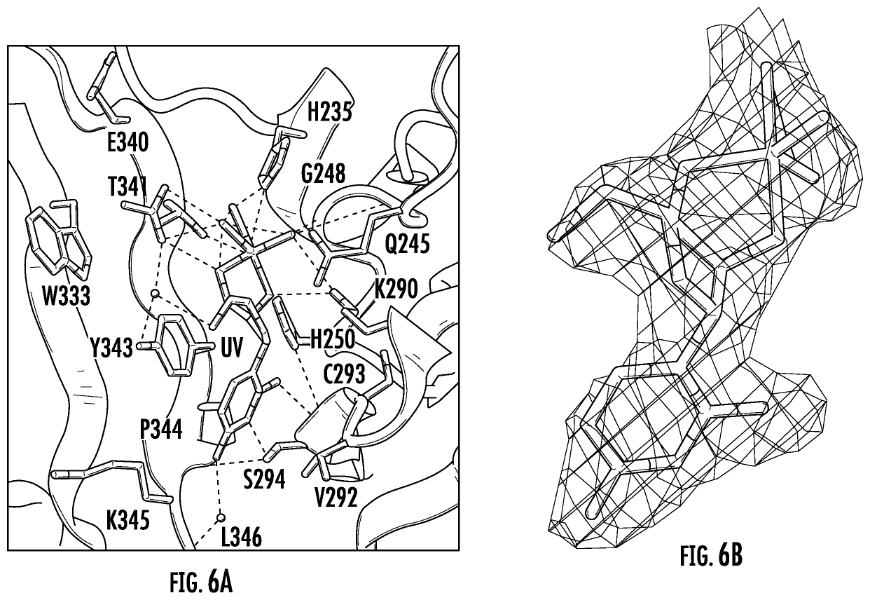

Direct structural evidence was found in the crystal structures for binding of each molecule listed in Table 1. Each molecule binds directly in the active site of Nsp15. FIGS. 1-6 show binding of tipiracil, 5′UMP, 3′UMP, GpU dinucleotide, citrate, and UV (a transition state analog), respectively, to the active site of Nsp15. With exception of the complex with UV, crystals with ligands diffract to higher resolution than the apoprotein (2.20 Å). Co-crystals of Nsp15 with 5′AMP (adenosine 5′-monophosphate), 3′,5′-cyclic AMP, 5′GMP (guanosine 5′-monophosphate), 5′TMP (thymidine 5′-monophosphate) and 5′CMP (cytidine 5′-monophosphate) using the same set of conditions as tested for 5′UMP could not be obtained.

All structures were solved by molecular replacement using Nsp15 (PDB id: 6WVV), the details of which are shown in Table 2.

TABLE 2

| | | | |
|---|---|---|---|
| Structure | Nsp15/5′UMP | Nsp15/3′UMP | Nsp15/5′GpU |

Data processing

| Space group | P6₃ | | P6₃ | | P6₃ | |
|---|---|---|---|---|---|---|
| Cell dimensions | 150.96, 111.30 | | 150.94, 111.86 | | 150.88, 111.79 | |
| a = b, c (Å) | 90, 90, 120 | | 90, 90, 120 | | 90, 90, 120 | |
| α, β, γ (°) | | | | | | |
| Resolution | 1.82 | (1.82-1.85) | 1.85 | (1.85-1.88) | 1.97 | (1.97-2.00) |
| range (Å)^{a} | | | | | | |
| Unique | 130,237 | (6,453) | 122,922 | (6,120) | 102,091 | (5,100) |
| reflections^{a} | | | | | | |
| R-merge^{b} | 0.148 | (1.879) | 0.146 | (1.587) | 0.155 | (1.452) |
| Mean I/sigma(I) | 18.5 | (1.0) | 25.1 | (1.5) | 16.0 | (1.30) |
| CC1/2^{c} | 0.985 | (0.407) | 0.999 | (0.561) | 0.972 | (0.373) |
| Completeness (%) | 100 | (100) | 100.0 | (99.7) | 100 | (99.8) |
| Redundancy | 7.5 | (6.3) | 12.8 | (6.4) | 6.2 | (4.8) |

| | | | |
|---|---|---|---|
| Wilson B- | 38.46 | 17.1 | 37.60 |
| factor (Å²) | | | |

Refinement

| | | | |
|---|---|---|---|
| Resolution | 1.82-40.63 | 1.85-45.19 | 1.97-49.39 |
| range (Å) | | | |
| Reflections | 123,356/6,406 | 108,782/5,676 | 96,700/5,021 |
| work/test | | | |
| R_{work}/R_{free} | 0.170/0.195 | 0.166/0.189 | 0.157/0.185 |
| Number atoms | | | |
| Protein | 5,707 | 5,700 | 5,540 |
| Ligand/ion | 125 | 81 | 102 |
| Water | 486 | 718 | 432 |
| Protein residues | 348 × 2 | 348 × 2 | 348 × 2 |
| RMSD | 0.006 | 0.002 | 0.002 |
| (bonds) (Å) | | | |
| RMSD | 0.762 | 0.534 | 0.405 |
| (angles) (°) | | | |
| Rotamer | 2.49 | 0.31 | 0.96 |
| outliers (%)^{d} | | | |
| Clashscore | 1.63 | 1.99 | 2.76 |
| Average | 48.8 | 25.6 | 47.8 |
| B-factor | | | |
| (Å²) | | | |
| Protein | 47.9 | 24.2 | 46.9 |
| Ligand/ion | 71.5 | 41.3 | 80.5 |
| Water | 52.8 | 33.7 | 51.0 |
| Number of | 10 | 7 | 13 |
| TLS groups | | | |

| Structure | Nsp15/tipiracil | | Nsp15/UV | |
|---|---|---|---|---|
| Data processing | | | | |
| Space group | P6₃ | | P6₃ | |
| Cell dimensions | 150.86, 111.70 | | 150.83, 110.73 | |
| a = b, c (Å) | 90, 90, 120 | | 90, 90, 120 | |
| α, β, γ (°) | | | | |
| Resolution | 1.85 | (1.85-1.88) | 2.25 | (2.25-2.29) |
| range (Å)^{a} | | | | |
| Unique | 122,260 | (5,907) | 66,979 | (3,067) |
| reflections^{a} | | | | |
| R-merge^{b} | 0.159 | (1.350) | 0.193 | (1.308) |
| Mean I/sigma(I) | 22.5 | (1.10) | 12.6 | (1.0) |
| CC1/2^{c} | 0.987 | (0.471) | 0.985 | (0.479) |
| Completeness (%) | 99.8 | (96.9) | 98.6 | (91.3) |
| Redundancy | 11.2 | (6.1) | 7.0 | (5.5) |

| Wilson B- | 30.97 | 42.0 |
|---|---|---|
| factor (Å²) | | |
| Refinement | | |
| Resolution | 1.85-42.45 | 2.25-49.37 |
| range (Å) | | |
| Reflections | 115,822/6,000 | 63,079/3,363 |
| work/test | | |
| R_{work}/R_{free} | 0.171/0.194 | 0.167/0.192 |
| Number atoms | | |
| Protein | 5,647 | 5,483 |
| Ligand/ion | 90 | 64 |
| Water | 702 | 265 |
| Protein residues | 348 × 2 | 348 × 2 |
| RMSD | 0.015 | 0.002 |
| (bonds) (Å) | | |
| RMSD | 1.251 | 0.494 |
| (angles) (°) | | |
| Rotamer | 2.68 | 0.65 |
| outliers (%)^{d} | | |
| Clashscore | 2.45 | 2.26 |
| Average | 41.1 | 50.7 |
| B-factor | | |
| (Å²) | | |
| Protein | 40.1 | 50.6 |
| Ligand/ion | 49.5 | 59.7 |
| Water | 48.5 | 50.3 |
| Number of | 10 | 9 |
| TLS groups | | |

^{a}Values in parentheses correspond to the highest resolution shell. The resolution was cut based on CC1/2 at higher than 0.35.
^{b}Rmerge = ΣhΣj|Ihj − <Ih>|/ΣhΣjIhj, where Ihj is the intensity of observation j of reflection h.
^{c}As defined by Karplus and Diederichs (Science 336, 1030-1033, (2012)).
^{d}As defined by Molprobity (Davis et al., Nucleic Acids Res. 32, W615-619, doi: 10.1093/nar/gkh39832/suppl_2/W615 [pii] (2004)).

The majority of the uncleaved His-tag residues were not ordered and hence not visible but for all residues from Met1 to Gln347 and for all bound ligands the electron density was excellent. In all five structures, the ligands bound to the C-terminal catalytic domain active site, though the exact positions vary, as described below. In the 5′GpU and tipiracil complexes, there was also a phosphate ion bound in the catalytic pocket. The compound binding was facilitated by side chains of seven conserved residues: His235, His250, Lys290, Trp333, Thr341, Tyr343, and Ser294 and main chain of Gly248, Lys345, and Val292, as well as water molecules. One non-conserved residue, Gln245, was also involved through a water-mediated interaction. The interactions do not trigger any major protein conformational changes either globally or locally. In fact, the catalytic residues (His235, His250) and other active site residues discussed below maintained very similar conformations in all complexes (RMSD 0.29 Å) over Cα atoms for residues His235, Gly248, His250, Lys290, Trp333, Thr341, Tyr343, and Ser294 in the pairwise superposition of complexes with the apo-structure (the highest is 0.39 Å for B chain of the tipiracil complex and the lowest is 0.29 Å for the chain B of the 5′UMP complex). The position of phosphate ion or 3′-phosphoryl group of 3′UMP was also preserved. The vanadate moiety of UV overlapped with the bound phosphate. Specific interactions of ligands with the protein are described below.

### 5′UMP Binding

The model of uracil binding by Nsp15 was proposed based on the EndoU and RNase A structures (Ulferts et al., RNA Biol 8, 295-304 (2011)), speculating that Ser294 might be responsible for base discrimination. The experimental details of pyrimidine recognition are as follows. The base of 5′UMP forms van der Waals contacts with Tyr343 and several hydrogen bonds with active site residues, including side chain OG and main chain nitrogen atom of Ser294. These interactions define O2 and N3 specificity. 04 interacts with the main chain nitrogen atom of Leu346 defining the uracil specificity. Potentially, cytosine and thymine pyrimidines may also bind—an amino group in position 4 of C should be compatible with the recognition pattern and a methyl group in position 5 should not interfere with binding either as uracil position 5 is solvent accessible. In fact, recognition of C has been reported for distantly related, though with similar active site, bacterial EndoU anticodon tRNase (Michalska et al., Mol Microbiol 109, 509-527 (2018)). The ribose ring makes several hydrogen bonds with protein residues: (i) 2′OH interacts with Lys290 (NZ) and with NE2 of the catalytic His250, (ii) 3′OH makes water mediated hydrogen bonds with catalytic His235 (NE2), His250 (NE2), Thr341 (OG1) and Gly248 (main chain nitrogen atom), and (iii) O4′ is hydrogen-bonded to main chain of Val292 via a water molecule. Interestingly, the 5′-phosphoryl group projects into solvent with no interaction with protein atoms. Its only ordered interaction is with 3′OH through a water molecule. This 5′-phosphoryl group location overlaps with that of Nsp15/5′GpU complex. The ribose in the Nsp15/5′UMP together with the phosphate ion in the Nsp15/tipiracil mimic 2′3′-cyclic phosphodiester. The structure with 5′UMP shows how the enzyme discriminates between uracil and purine bases with Ser294 serving as the key discriminatory residue, as has been hypothesized before (Bhardwaj et al., J Mol Biol 361, 243-256 (2006), Bhardwaj et al., J Virol 78, 12218-12224 (2004); Bhardwaj et al., J Biol Chem 283, 3655-3664 (2008); and Ivanov et al., Proc Natl Acad Sci USA 101, 12694-12699 (2004)).

### 5′GpU Binding

In RNA sequence containing uracil, such as 5′NpGpUpN3′, where N corresponds to any base, the Nsp15 cleavage would produce 5′NpGpU3′p, if transphosphorylation is followed by hydrolysis of 5′NpGpU2′3′p. In the crystal structure of Nsp15/5′GpU, the dinucleoside monophosphate binds to the active site with uracil interacting with Tyr343 and Ser294, as seen in the Nsp15/5′UMP complex. However, the distance between 04 and the main chain nitrogen of Leu346 is too long to make a hydrogen bond. This implicates some flexibility at the protein C-terminus and suggests that the amino group in position 4 of cytosine can be accommodated, as suggested above and reported previously (Kim et al., Protein science: a publication of the Protein Society, doi:10.1002/pro.3873 (2020)). The guanine ring is stacking against Trp333 and makes two hydrogen bonds with water molecules. The absence of defined base-side chain interactions suggests lack of specificity for this site in the substrate sequence. The Nsp15/5′GpU complex binds also a phosphate ion in the active site, most likely from the crystallization buffer. The ion interacts with the protein side chains (His235, His250, Thr341, and Lys290) and uridine ribose 2′OH and 3′OH groups. It most likely mimics the binding of scissile phosphoryl group of the substrate. The backbone phosphoryl group (5′ of U) faces solvent as in the Nsp15/5′UMP complex and makes a hydrogen bond to a water molecule. This structure and the Nsp15/5′UMP complex illustrate location and specificity determinants of the uridine with a 5′-phosphoryl group. The binding of guanine in the structure identifies strong base binding site at Trp333, which, however, may not necessarily be dedicated to 5′end of the oligonucleotide.

### 3′UMP Binding

Nsp15 was co-crystallized with 3′UMP, assuming that the enzyme would dock it in a manner expected for uridine monophosphate nucleotide in the contexts of larger RNA substrate, preserving the uracil specific interactions described above. Surprisingly, the uracil base is anchored by Trp333 in the guanine site observed in the Nsp15/5′GpU complex, confirming that this site can accommodate purine and pyrimidine bases. The 3′-phosphoryl group occupies the phosphate ion site created by His235, His250, and Thr341, as observed in Nsp15/5′GpU. It is surprising that uracil does not go into its dedicated site, but the result demonstrates higher affinity for the base in the Trp333 site than in uracil-recognition site, potentially governed by the strong stacking interactions with the aromatic side chain that take precedence over hydrogen bonds observed in the uracil binding of 5′UMP. The identity of the Trp333-interacting base is irrelevant, especially given that the enzyme's substrate is most likely a larger RNA molecule.

### Tipiracil Binding—a Uracil Derivative and Nsp15 Inhibitor

The tipiracil molecule binds to the uracil site as observed in 5′UMP and 5′GpU. The molecule makes several substrate analog-like interactions. The uracil ring stacks against Tyr341 and makes hydrogen bonds with Ser294 (interacting with O2 and N3), Lys345 (O4), and His250. The carbonyl group of Leu346 makes a water-mediated hydrogen bond in the structure with tipiracil. The N1 atom makes a hydrogen bond with a phosphate ion and through it connects to Lys290. There are also two water-mediated interactions to Ser294 and main chain carbonyl oxygen atom of Val292. The iminopyrrolidine nitrogen atom binds to Gln245, representing the only interaction unique to the ligand. Nsp15 binds tipiracil in its active site in a manner compatible with competitive inhibition, and the compound and its derivatives are expected to serve as inhibitors of the enzyme.

### Binding of Uridine 2′,3′-Vanadate—S_{N}2 Transition State Analog

The structure of the complex with UV was determined at 2.25 Å resolution. UV binds to the uridine binding site as observed in the structures with 5′UMP, GpU, and tipiracil. The uracil moiety is recognized by Ser294 and Leu346 defining the uracil recognition and interacts also with Tyr343. The pentavalent vanadate is covalently bound to 2′ and 3′ ribose oxygen atoms, and it corresponds to the S_{N}2 transition state (Davies et al., FEBS Lett 577, 315-321 (2004); Deng et al., Journal of the American Chemical Society 120, 4717-4722 (1998); Messmore et al. J Am Chem Soc 122, 9911-9916 (2000); Veenstra et al., Biophys J 67, 331-335 (1994); and Wlodawer et al., Proc Natl Acad Sci USA 80, 3628-3631 (1983)). The metal ion, O3′ and two vanadate oxygen atoms are in a plane. One oxygen (O2′) atom below the plane is interacting with His250 and the other oxygen above the plane is in contact with His235. The vanadate position is consistent with 3′-phosphoryl group that is cleaved in the RNA substrate and observed in the GpU and tipiracil complexes. The vanadate interacts with key catalytic site residues including His250, His235, Lys290, Thr341, and Gly247 and has several water-mediated interactions. Particularly, Lys290 makes hydrogen bonds to O2′ and one of the vanadate oxygen atoms in the plane reminiscent of Lys41 in RNase A. These interactions confirm previous experimental observations for Nsp15 and are consistent with the proposed S_{N}2 mechanism. The structure also provides insights into the structure of 2′,3′-cyclic phosphodiester, which is the product of transphosphorylation reaction. This structure suggests that Nsp15 should follow a two-step reaction mechanism with the final product being 3′UMP.

### Example 2

This example describes the influence of inhibitors on Nsp15 thermal stability in an embodiment of the invention.

The Nsp15 differential scanning fluorimetry (DSF) assays were performed in a buffer 20 mM Tris pH 7.5, 100 mM NaCl, 1 mM TCEP that was supplemented with SYPRO™ Orange protein gel stain (Invitrogen, Carlsbad, Calif.) to 5× final concentration. 10 μM of the Nsp15 was used with the addition of each of 1 mM of tipiracil, 5′GpU, 5′UMP, 3′UMP, and TMP to get final molar ratio of protein to ligand 1:100. Additionally, the samples with ligands were supplemented with 10 mM MnCl₂. After 30 min of incubation of samples at room-temperature, fluorescence measurements were done using CFX CONNECT™ Real-Time System (BioRad, Hercules, Calif.). Fluorescent signal was detected with temperature rate 1° C. per 60 sec. Samples were measured in triplicate, and the representative standard deviation of measurements are depicted for Nsp15 control sample and complex of the Nsp15 with addition of tipiracil.

The differential scanning fluorimetry experiments (DSF) showed that the melting temperature (Tm) of the Nsp15 in a presence of tipiracil, 5′GpU, 5′UMP, 3′UMP and 3′TMP is approximately 60.5° C. under investigated conditions with buffer that contains 10 mM MnCl₂. Depicted local minima of the Nsp15 first derivative of fluorescence signals have the same Tm values as a control. The denaturation profile of the Nsp15 in the presence of tipiracil is broader and consistently shifted (0.5° C.) to higher temperatures. Therefore, DSF results indicate the small increase of stability of the Nsp15/tipiracil complex in comparison to control sample and other tested Nsp15 complexes with 5′GpU, 5′UMP, 3′UMP and TMP. Additional change in the Nsp15 Tm was observed at 83° C. This is caused by all ligands and may be related to increased stability of the hexamer or EndoU catalytic domain. In the presence of Mn²⁺ ions, the main Tm of Nsp15 was increased from 56.5° C. to 60.5° C. and is Mn²⁺ concentration dependent. At 5 mM and 20 mM concentrations of Mn²⁺, a new local Tm minimum was observed at 83° C. potentially suggesting increased stability of the EndoU domain.

### Example 3

This example demonstrates inhibition of Nsp15 activity by tipiracil.

### Radioactivity-Based Endoribonuclease Assays

A typical reaction contained 5×10⁵ CPM of 5′-³²P-labeled RNA eicosamers or 3′-³²P-labeled RNA octamers (0.5 μM final RNA concentration) and 10 nM SARS-CoV-2 Nsp15 in 20 mM 4-(2-hydroxyethyl)-1-piperazineethanesulfonic acid-KOH (HEPES-KOH) buffer (pH 7.5), 50 mM KCl, 1 mM dithiothreitol (DTI) and either 5 mM or 15 mM MnCl₂. To inhibit endonuclease, tipiracil dissolved in water was added. Following incubation at 30° C. for up to 60 min, the reactions were stopped by adding an equal volume of a gel-loading buffer containing 95% formamide, 10 mM ethylenediaminetetraacetic acid (EDTA), and 0.025% sodium dodecyl sulfate (SDS). The products were analyzed in 20% polyacrylamide gels (acrylamide/bisacrylamide ratio of 19:1) buffered with 0.5× Tris-borate-EDTA containing 7M urea.

For cleaving pCp and pUp, a typical reaction contained 5×10⁵ CPM of 10 μM [5′-32P]pCp and [5′-³²P]pUp. To inhibit endonuclease, tipiracil dissolved in dimethylsulfoxide (DMSO) was added (5% final DMSO concentration). The products of Nsp15 reaction with [5′-³²P]pCp and [5′-³²P]pUp were separated by chromatography on polyethylenimine (PEI)-cellulose thin-layer plates in 0.1 M ammonium acetate and 5% acetic acid as described by Bullock et al., J. Mol. Biol., 328(2): 395-408 (2003), incorporated herein by reference in its entirety.

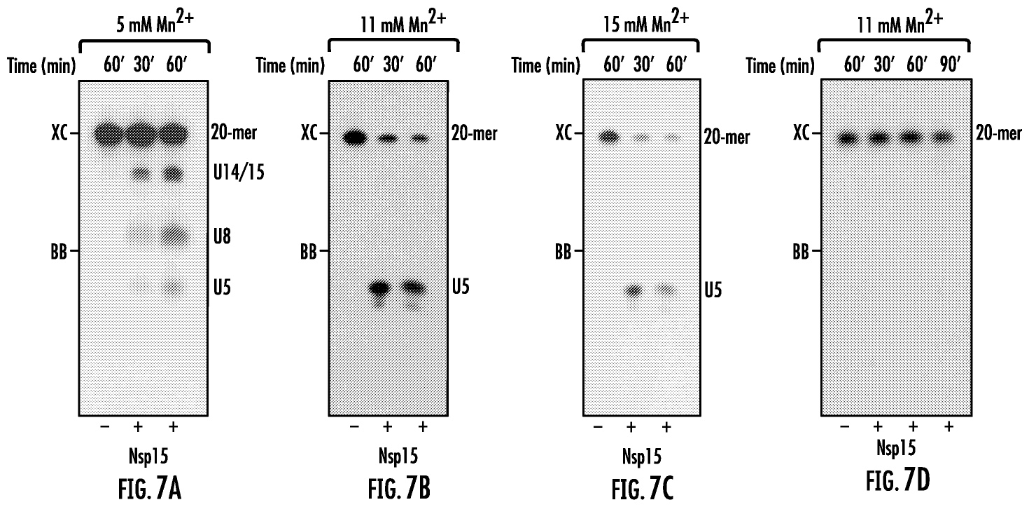

FIGS. 7A-D show cleavage of a radiolabeled ribonucleotide of radiolabeled GAACUCAUGGACCUUGGCAG (SEQ ID NO: 1) (FIGS. 7A-C) and GAACACAAGGACCAAGGCAG (SEQ ID NO: 2) by Nsp15. The cleavage of the eicosamer is uridine-specific and show no sequence preference as 5′CU↓C, 5′AU↓G, 5′CU↓U and 5′UU↓G are recognized and cut, especially at higher manganese concentration (compare FIGS. 7A and 7B). At 5 mM manganese, all four oligonucleotides accumulate, with the slowest cut sequence being the 5′AU↓G site (FIG. 7A). The eicosamer that does not contain uridine, SEQ ID NO: 2, is not cut (FIG. 7D).

The observed CoV-2 Nsp15 Mn²⁺ dependence is unlike its SARS-CoV homolog (Nedialkova et al., J Virol 83, 5671-5682 (2009)). This is surprising as SARS-CoV-2 Nsp15 shares 88% sequence identity and 95% similarity with the SARS-CoV homolog and all active site residues are conserved. Without wishing to be bound by theory, one explanation would be that the metal ion impacts the structure of RNA and the cleavage rate will depend on the sequence (and structure) of the substrate. Interestingly, by itself the Nsp15 can cut RNAs at any uridine site, but as a component of replicase-transcriptase complex (RTC) with Nsp8 and Nsp12, Nsp15 becomes a site specific endonuclease which cuts RNA to leave short 5-10 uridine tails (Hackbart et al., Proc Natl Acad Sci USA 117, 8094-8103 (2020)). The enzyme also cuts the 3′ end of conserved non-coding region of RNA (Bhardwaj et al., J Mol Biol 361, 243-256, (2006); and Gioia et al., J Biol Chem 280, 18996-19002 (2005)), and the cleavage is RNA secondary structure dependent.

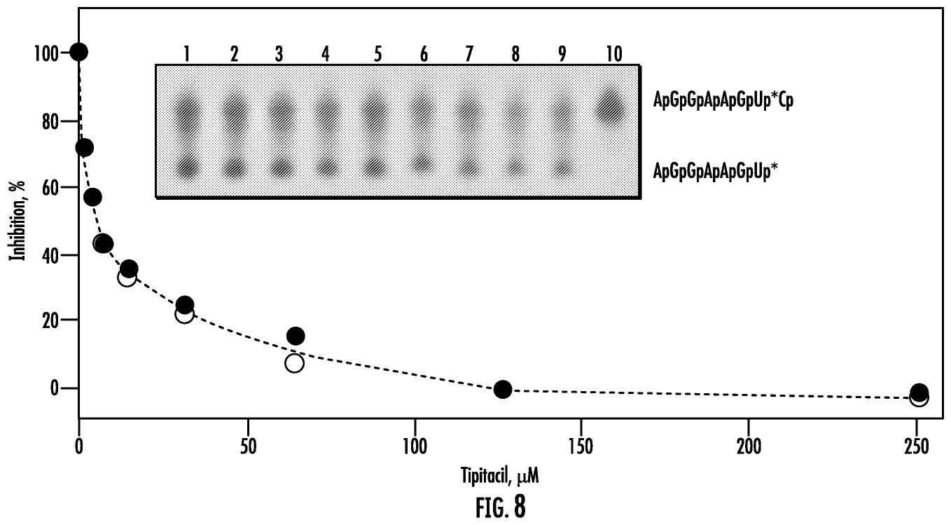

Octamer RNA with single uridine site 5′AGGAAGU³²pC (SEQ ID NO: 3), which under experimental conditions remains single-stranded and does not form duplexes. Upon endonuclease reaction, 3′-³²P-labeled heptamer and unlabeled 3′CMP are produced. The transfer of the ³²P from the pCp to the 3′ terminus of the heptamer is consistent with transphosphorylation mechanism. In the presence of 5 mM MnCl₂, only partial cleavage was observed. This reaction is decreased by 50% in the presence of 7.5 μM tipiracil (FIG. 8). At the higher Mn²⁺ ion concentration, the cleavage reaction proceeded to completion but no inhibition by tipiracil can be measured.

### Fluorescence-Based Endoribonuclease Assays

The substrate used in the endoribonuclease assay possesses carboxyfluorescein (FAM) at the 5′ terminus of the oligonucleotide and quencher molecule tetramethylrhodamine (TAMRA) at the 3′ end. The oligonucleotide dArUdAdA with 5′-6-FAM and 3′-6-TAMRA was synthetized by GenScript Biotech (Piscataway, N.J.). The concentration of 0.5 μM substrate was used with 2 μM Nsp15 in a buffer containing 20 mM HEPES at pH 7.8, 100 mM NaCl, and 5 mM MnCl₂. Detection of the emission of FAM fluorescence signal was done on CFX CONNECT™ Real-Time System (Bio-Rad Laboratories, Hercules, Calif.).

Cleavage of oligonucleotide dArUdAdA with 5′-6-FAM and 3′-6-TAMRA substrate was tested using polyacrylamide gel electrophoresis. The concentration of labeled substrate was 10 M, and the Nsp15 concentration varied to achieve different molar ratios of substrate to Nsp15. Samples were mixed in a buffer with 20 mM HEPES at pH 7.8, 100 mM NaCl, and 5 mM MnCl₂. After 10 minutes of incubation, samples were loaded on NUPAGE Tris-Acetate 3-8% gel, and the electrophoresis was done on 80 V for 1 h. The detection of FAM emission on gel was performed using the Gel DOC™ EZ System (Bio-Rad Laboratories, Hercules, Calif.).

It was found that 50% inhibition occurred at 72 micromolar concentration of tipiracil.

Comparison of the CoV-2 Nsp15 active site with RNase A inhibition of Nsp15 activity by tipiracil can provide information as to the catalytic mechanism of Nsp15 endoribonuclease. Structures were compared with eukaryotic RNase A, a very well-studied model system (Cuchillo et al., Biochemistry 50, 7835-7841 (2011)). RNase A recognizes pyrimidine nucleotides in RNA, preferring C over U, and catalyzes a two-step reaction, the transphosphorylation of RNA to form a 2′,3′-cyclic CMP intermediate followed by its hydrolysis to 3′CMP. In RNase A the base selectivity is achieved by Thr45 that forms specific hydrogen bonds similar to those created by Ser294 in the Nsp15/5′UMP, Nsp15/tipiracil, and Nsp15/5′GpU complexes. In RNase A, the transphosphorylation reaction proceeds via an asynchronous concerted general acid/base mechanism involving His12, His119, and Lys41 (Harris et al., Biochim Biophys Acta 1854, 1801-1808 (2015)). In this mechanism the 2′OH proton is transferred to the deprotonated form of His12 to activate the 2′O nucleophile. Then, the protonated His119 donates a proton to the departing 5′OH group. Lys41 function is to stabilize the negative charge that accumulates on the nonbridging phosphoryl oxygen atoms in the transition state. In the hydrolysis step, the role of two histidine residues are inverted. The Nsp15 mechanism of catalysis is consistent with that of RNase A as observed in the structure of the complex with UV transition state. The 2′OH proton is transferred to the deprotonated form of His250 to activate the 2′O nucleophile, the protonated His235 donates a proton to the departing 5′OH group and Lys290 stabilizes the negative charge of the vanadate ion.

The RNase A active site is well organized and has several distinct pockets for binding RNA substrates (e.g. bases B1, B2, B3 and phosphoryl groups P0, P1 and P2) (Nogues et al., Biochim Biophys Acta 1253, 16-24 (1995); and Raines, R. T., Chem Rev 98, 1045-1066 (1998)). The B1 site provides base specificity and P1 site binds the scissile phosphoester bond. B0 represent the binding site for the base upstream of the B1-P1 cleavage site. In the RNase A complex with the 5′dApdTp↓dApdA deoxyoligonucleotide (PDB id: 1RCN; Fontecilla-Camps et al., J Biol Chem 269, 21526-21531, (1994)), B0 interacts with adenine, B1 binds thymine, etc. The P1 site represents the above-mentioned catalytic machinery consisting of His12, His119, and Lys41 and Lys41 assisted by Gln11 in phosphoryl binding.

Structural alignment of the Nsp15 and RNase A catalytic site residues and RNA ligands shows that they adopt similar positions in the two enzymes, despite sequence and structure dissimilarity. Specifically, His250, Ser294, and Lys290 virtually overlap with the His12, Thr45, and Lys41. His250, by analogy His12 in RNase A, is in position to serve as the key residue in deprotonation of 2′OH. It is close to 2′OH in both 5′GpU and 5′UMP structures (3.2 and 3.8 Å respectively) and is in similar orientation in the complex of RNase A with the DNA substrate analog. Therefore, His250 is very likely to directly activate 2′OH. Lys290 seems to play a role of Lys41 in RNase A. Main chain amide of Gly248 may provide function of Gln11 in binding to the substrate phosphoryl group. If His250 is a base in Nsp15 then the His235 must be a proton donor for the departing 5′OH group and equivalent of His119 of RNase A. However, these residues are ˜8 Å apart and they approach the phosphoryl group from different directions. In RNase A, His119 forms a hydrogen bond with Asp121 which may provide proton for the reaction. The structural environment for His235 is different in Nsp15. Thr341 forms a hydrogen bond with His235 and there is also Asp240 further away that makes a water-mediated hydrogen bond with His235. In the hydrolysis step of converting the 2′3′-cyclic phosphate back to 2′OH and 3′-phosphoryl group, the roles of histidine residues are reversed and now His235 must be a base deprotonating a water molecule and His250 serves as a proton donor for the 2′OH leaving group. A different set of interactions involving Nsp15 and RNase A catalytic residues may explain why Nsp15 activity is more sensitive to low pH and it is expected that kinetics of the reactions will be different.

Besides similarities in P1, the two enzymes share the organization of B1 pocket. Here, Nsp15 has a very well-defined uracil recognition site made of Ser294, Tyr343, and Leu346 that are equivalent to Thr45, Phe120, and Ser123 in RNase A. Further extrapolation from the RNase A model of the deoxyoligonucleotide binding allows the hypothesis that the B2 site, dedicated to the base on the 3′ end of scissile bond, in Nsp15 is created by Trp333. Its side chain provides stacking option for a base with no base selectivity function. Yet in the Nsp15/5′GpU structure, this site is occupied by the G base located on the 5′end of U. It is speculated that for oligonucleotides that are flanked on both sides of U, mimicking the RNase A ligand, 5′ guanine (or a different base) may adopt position of 5′dA that locates to the B0 site owing to the rotation of the P-5′O bond. Then, the available Trp333 base binding site can accept base on the 3′ end of the uridine moiety, somewhat resembling the Nsp15/3′UMP structure. When oligonucleotides are combined from the Nsp15 structures with RNase A/DNA complex, a plausible model can be built of 5′GpUp↓U nucleotide bound to the Nsp15 active site. This model underscores the importance of conserved Trp333 in anchoring RNA in the active site. While Ser294 is the key residue in discriminating base, the hydrophobic interaction with Trp333 may be a significant force for ligand docking. The B3 site is not easily identifiable in the available Nsp15 structures. In addition, unlike in RNase A, where all P0-P2 subpockets contribute to the backbone binding, in Nsp15 only the P1 site is currently well defined. 5′-phosphoryl groups in position P0 of the ligands do not form any direct contacts with the protein, while RNase A P0 site has Lys66 participating in RNA binding. The P2 site of RNase A is created by Lys7 and it appears that His243 may fulfill such role in Nsp15.

### Example 4

This example describes molecule screening of SARS-CoV-2 in an embodiment of the invention.

All SARS-CoV-2 infections are performed in biosafety level 3 conditions. VeroE6 and hACE2-A549 cells expressing human ACE2 were separately treated with no active agent and an active agent at 1, 10, and 50 μM for 2 hours in triplicate for each assay. The active agents were selected from 3′-uridylic acid, 5′-uridylic acid, citrate, GpU dinucleotide or derivatives thereof of formula (I), and tipiracil or N-substituted derivatives of formula (II), as described herein. Cells were infected with 0.1 multiplicity of infection SARS-CoV-2 (nCoV/Washington/1/2020, provided by National Biocontainment Laboratory, Galveston, Tex.) for 2 hours. Virus inoculum is then removed and replaced with media containing the appropriate concentration of active agent. At 24 hours, the infection is harvested for the following assays:

1. Viral RNA quantitation. Cell supernatants are collected and used for infectivity quantitation as described below. RNA is then isolated from infected cells via 96-well RNA isolation plates by standard protocol. Viral RNA is quantified by a standard CDC-verified primer/probe using quantitative qRT-PCR (quantitative reverse transcriptase polymerase chain reaction) set: F—5′-GAC CCC AAA ATC AGC GAA AT-3′ (SEQ ID NO: 4), R—5′-TCT GGT TAC TGC CAG TTG AAT CTG-3′ (SEQ ID NO: 5), Probe: 5′-ACC CCG CAT TAC GTT TGG TGG ACC-3′ (SEQ ID NO: 6) having 5′FAM (5-carboxyfluorescein) and 3′BAM fluorescent labels. O: 5) having 5′FAM (5-carboxyfluorescein) and 3′FAM (3-carboxyfluorescein) fluorescent labels. These are amplified under standard conditions with the HTRL STEPONEPLUS™ 96-well Real-Time PCR (polymerase chain reaction) System (ThermoFisher Scientific, Waltham, Mass.). A multiplex assay is used with 18S RNA as an internal control. Percent viral replication is calculated and plotted against active agent concentration to give EC₅₀ and EC₉₀ values for the active agent.

Percent viral replication is lower in samples that are in the presence of an active agent compared to samples that have no active agent.

2. Viral infectivity assay (plaque assay). Cell supernatant from above are diluted 10-fold serially and used to infect 6-wells of VeroE6 cells for 1 hour; inoculum is removed, and warm oxoid agar/media is added and incubated 3 days. Plates are fixed in methanol +5% formaldehyde overnight and stained with crystal violet for 1 h and counted. This is used as a primary tool for quantifying virus stocks, described as plaque forming units (PFU)/mL.

The PFU/ml value is lower in samples that are in the presence of an active agent compared to samples that have no active agent.

3. Viral antigen assay. Cells are infected as above and then fixed with 4% paraformaldehyde, blocked, then probed with mouse anti-Spike antibody for 4 hours, rinsed and probed with anti-mouse-HRP for 1 hour, rinsed and developed with 3,3′-diaminobenzidine tetrahydrochloride (DAB) substrate for 10 minutes. Colorimetric changes are quantified by absorbance using a 96-well plate reader.

The quantified value is lower in samples that are in the presence of an active agent compared to samples that have no active agent.

4. Cell toxicity assay. In parallel, uninfected cells are treated with the active agent as above and after 24 hours cell viability is measured using CELLTITER GLO™ luciferase assay (Promega, Madison, Wis.). The 50% cytotoxicity level (CC₅₀) is quantified and a selectivity index (EC₅₀:CC₅₀) is calculated. Molecules with a selectivity index >50 are prioritized for follow up.

5. Whole Cell Assay 1—Immunostaining against spike protein. Cell viability was measured by staining with CELLTRACKER™ Red CMTPX (ThermoFisher Scientific, Waltham, Mass.). CoV-2 infected cell was stained with 2 μM of CELLTRACKER™ Red CMTPX for 30 min and then detected by ELISA microplate reader Tecan Infinite m200 (Tecan, Switzerland) at ex577/em602 nm. After reading, cells were fixed by 10% neutral buffered formalin (NBF) for the following immunohistochemistry assay.

Immunostaining was performed on 10% NBF fixed SARS-CoV-2 infected cells in 96-well plate. After fixation, 10% NBF was removed, and cells were washed with phosphate-buffered saline (PBS), followed by washing with PBS-T (0.1% TWEEN™ 20 in PBS) (Sigma Aldrich, St. Louis, Mo.), and then blocked for 30 min with PBS containing 1% bovine serum albumin (BSA) at room temperature. After blocking, endogenous peroxidases were quenched by 3% hydrogen peroxide for 5 min. Then, cells were washed with PBS and PBS-T and incubated with a monoclonal mouse-anti-SARS-CoV-2 spike antibody (1:1000, GeneTex, Irvine, Calif.) in PBS containing 1% BSA overnight at 4° C. Primary antibody was washed with PBS and PBS-T and then cells were incubated in secondary antibody (IMMPRESS™ Horse Anti-Mouse IgG Polymer Reagent, Peroxidase; Vector Laboratories, Burlingame, Calif.) for 60 min at room temperature. After washing with PBS for 10 min, color development was achieved by applying diaminobenzidine tetrahydrochloride (DAB) solution (Metal Enhanced DAB Substrate Kit; ThermoFisher Scientific, Waltham, Mass.) for 30 min and detected by Tecan Infinite m200 (Tecan, Switzerland) at 492 nm after replacing DAB solution to PBS. Result was normalized by cell viability.

6. Whole cell assay 2—RNA Extraction and qRT-PCR. Total RNA from SARS-CoV-2 infected cells was isolated using a NucleoSpin 96 RNA kit following the manufacturer's instructions (Macherey-Nagel, Germany). SARS-CoV-2 RNA was quantified by qRT-PCR using SUPERSCRIPT™ III PLATINUM™ One-Step qRT-PCR Kit w/ ROX (ThermoFisher Scientific, Waltham, Mass.) and normalized using Eukaryotic 18S rRNA Endogenous Control (VIC™/MGB probe, Applied Biosystems,) via a STEPONEPLUS™ real-time PCR system (Applied Biosystems, Foster City, Calif.). All reaction performed in a dual-plex qRT-PCR using the Centers for Disease Control and Prevention (CDC) recommended primers for N1. Primer and probe sequences are as follows: 2019-nCoV_N1 Forward Primer (2019-nCoV_N1-F), GACCCCAAAATCAGCGAAAT (SEQ ID NO: 7); 2019-nCoV_N1 Reverse Primer (2019-nCoV_N1-R), TCTGGTTACTGCCAGTTGAATCTG (SEQ ID NO: 8); Probe (2019-nCoV_N1-P), FAM-ACCCCGCATTACGTITGGTGGACC-BHQ1 (SEQ ID NO: 9).

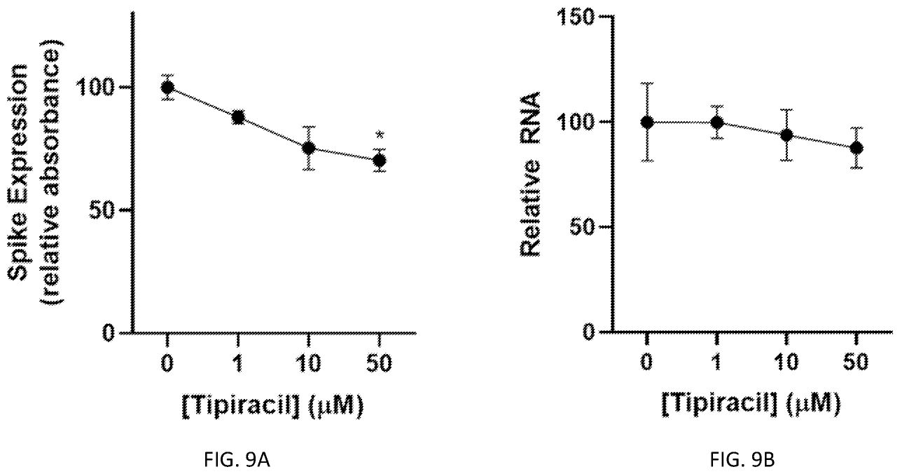

Tipiracil did not affect the viability of cells, but the inhibition of virus was found to be modest in the concentration range of 1-50 μM (FIGS. 9A-B).

### Example 5

This example describes in vivo testing in an embodiment of the invention.

Two mammals are infected with a nidovirus that encodes Nsp15. An active agent selected from 3′-uridylic acid, 5′-uridylic acid, citrate, GpU dinucleotide or derivatives thereof of formula (I), and tipiracil or N-substituted derivatives thereof of formula (II) as described herein is administered to one of the mammals. The mammal administered the active agent experiences inhibition of the infection as measured by reduced severity of one or more symptoms, reduced virus titer, or other standard measurement, compared to the mammal not administered the active agent.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a disease caused by a nidovirus that encodes Nsp15 in a subject in need thereof comprising administering to the subject a therapeutically effective amount of an active agent selected from 3′-uridylic acid, 5′-uridylic acid, citrate, a tetracycline antibiotic of the formula 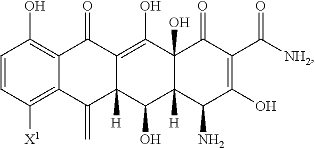
wherein X¹ is H or Cl and salts thereof,
an anthracycline antineoplastic agent with a core structure of 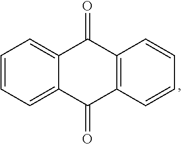 [SMILES: O=C1c2ccccc2C(=O)c2ccccc21]
sorafenib, sunitinib malate, primaquine diphosphate, closantel,
a GpU dinucleotide of formula (I): 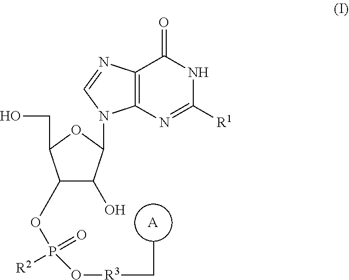
wherein
ring A is 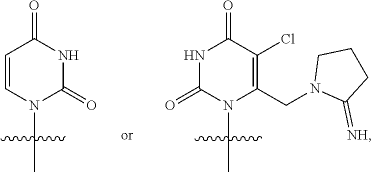 [SMILES: *n1ccc(=O)[nH]c1=O ; *n1c(CN2CCCC2=N)c(Cl)c(=O)[nH]c1=O]
R¹ is —NR⁴R⁵,
R² is O⁻M⁺ or —(CR⁶R⁷)ₙ—R⁸,
R³ is 1′,5′-ribosyl of the formula 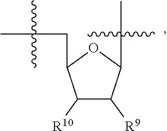
—CH₂CH₂CH₂CH₂—, —CH₂CH₂CH(CO₂H)CH₂—, or —CH₂CH(CO₂H)CH₂CH₂—,
R⁴ and R⁵ is the same or different and each is H or alkyl,
each instance of R⁶ and R⁷ is the same or different and each is H or alkyl,
R⁸ is alkyl, —OR¹¹, —SR¹¹, or —NR⁴R⁵, and
R⁹ and R¹⁰ are the same or different and each is —(CR⁶R⁷)ₘ—R⁸, or R⁹ and R¹⁰ together form —(CR⁶R⁷)ₒ—, and
R¹¹ is H or alkyl,
M⁺ is a counterion,
m and n are the same or different and each is 0 or an integer of 1 to 4, and
is 3 or 4, and
a compound of formula (II): 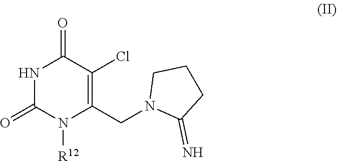
wherein
R¹² is H or —(CR¹³R¹⁴)ₙ—R¹⁵,
each instance of R¹³ and R¹⁴ is the same or different and each is H or alkyl,
R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,
R¹⁶ and R¹⁸ is the same or different and each is H or alkyl,
R¹⁷ is H, alkyl, OH, or SH,
X is O, S, or NR⁵, and
n is 0 or an integer of 1 to 4,
or a pharmaceutically acceptable salt thereof.

2. A method of inhibiting an Nsp15 endoribonuclease of a nidovirus that encodes Nsp15 comprising contacting the Nsp15 endoribonuclease with an effective amount of an active agent selected from 3′-uridylic acid, 5′-uridylic acid, citrate,
a tetracycline antibiotic of the formula 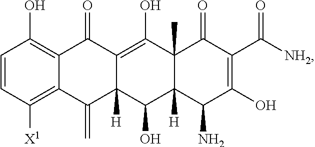
wherein X¹ is H or Cl and salts thereof,
an anthracycline antineoplastic agent with a core structure of 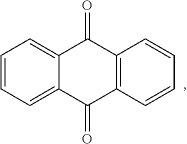 [SMILES: O=C1c2ccccc2C(=O)c2ccccc21]
sorafenib, sunitinib malate, primaquine diphosphate, closantel,
a GpU dinucleotide of formula (I): 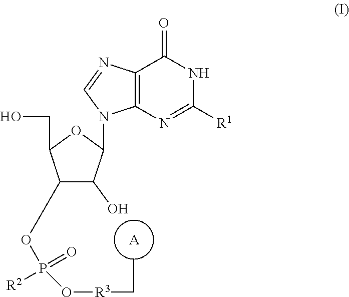
wherein
ring A is 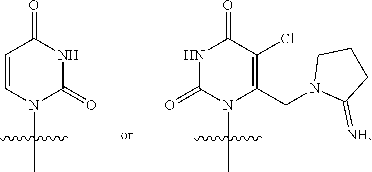 [SMILES: *n1ccc(=O)[nH]c1=O ; *n1c(CN2CCCC2=N)c(Cl)c(=O)[nH]c1=O]
R¹ is —NR⁴R⁵,
R² is O⁻M⁺ or —(CR⁶R⁷)ₙ—R⁸,
R³ is 1′,5′-ribosyl of the formula 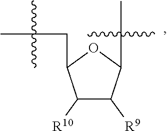
—CH₂CH₂CH₂CH₂—, —CH₂CH₂CH(CO₂H)CH₂—, or —CH₂CH(CO₂H)CH₂CH₂—,
R⁴ and R⁵ is the same or different and each is H or alkyl,
each instance of R⁶ and R⁷ is the same or different and each is H or alkyl,
R⁸ is alkyl, —OR¹¹, —SR¹¹, or —NR⁴R⁵, and
R⁹ and R¹⁰ are the same or different and each is —(CR⁶R⁷)ₘ—R⁸, or R⁹ and R¹⁰ together form —(CR⁶R⁷)ₒ—, and
R¹¹ is H or alkyl,
M⁺ is a counterion,
m and n are the same or different and each is 0 or an integer of 1 to 4, and
o is 3 or 4, and
a compound of formula (II): 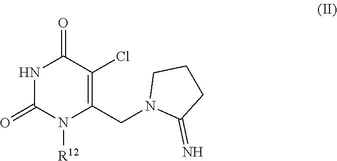
wherein
R¹² is H or —(CR¹³R¹⁴)ₙ—R¹⁵,
each instance of R¹³ and R¹⁴ is the same or different and each is H or alkyl,
R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,
R¹⁶ and R¹⁸ is the same or different and each is H or alkyl,
R¹⁷ is H, alkyl, OH, or SH,
X is O, S, or NR⁵, and
n is 0 or an integer of 1 to 4,
or a pharmaceutically acceptable salt thereof.

3. The method of claim 1, wherein the active agent is 3′-uridylic acid or 5′-uridylic acid.

4. The method of claim 1, wherein the active agent is citrate, sorafenib, sunitinib malate, primaquine diphosphate, closantel,
a tetracycline antibiotic of the formula 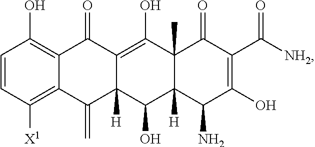
wherein X¹ is H or Cl and salts thereof, or
an anthracycline antineoplastic agent with a core structure of

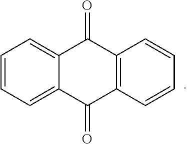 [SMILES: O=C1c2ccccc2C(=O)c2ccccc21]

5. The method of claim 1, wherein the active agent is a GpU dinucleotide of formula (I).

6. The method of claim 5, wherein ring A is 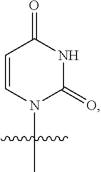 [SMILES: *n1ccc(=O)[nH]c1=O]
R¹ is NH₂, R² is O⁻Na⁺, R³ is 1′,5′-ribosyl of the formula 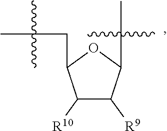
and R⁹ and R¹⁰ are both OH.

7. The method of claim 1, wherein the active agent a compound of formula (II) or a pharmaceutically acceptable salt thereof.

8. The method of claim 7, wherein R¹² is H or a pharmaceutically acceptable salt thereof.

9. The method of claim 7, wherein R¹² is or —(CR¹³R¹⁴)ₙ—R¹⁵, R¹⁵ is —OR¹⁶, —NR¹⁶R¹⁷, —C(O)OR¹⁶, —C(O)—NR¹⁶R¹⁷, or —NR¹⁶C(O)OR¹⁸, and n is in integer of 1 to 4, or a pharmaceutically acceptable salt thereof.

10. The method of claim 9, wherein R¹³ and R¹⁴ are each H, R¹⁶ is H or methyl, R¹⁷ is H, methyl, or OH, R¹⁸ is methyl, and n is an integer of 2 or 3, or a pharmaceutically acceptable salt thereof.

11. The method of claim 7, wherein R¹² is —(CH₂)₂OH, —(CH₂)₃OH, —(CH₂)₂OCH₃, —(CH₂)₃OCH₃, —(CH₂)₂NH₂, —(CH₂)₂N(CH₃)₂, —(CH₂)₂CO₂CH₃, —(CH₂)₃CO₂CH₃, —(CH₂)₄CO₂CH₃, —(CH₂)₂NHCO₂H, or —(CH₂)₂NHCO₂CH₃, or a pharmaceutically acceptable salt thereof.

12. The method of claim 8, wherein the compound of formula (II) is Crystal Form I, exhibiting powder X-ray peaks at two or more angles selected from the group consisting of 11.6°, 17.2°, 17.8°, 23.3°, 27.1°, and 29.3° as a diffraction angle (2θ±0.2°).

13. The method of claim 12, wherein the compound of formula (II), wherein the compound of formula (I) is 5-chloro-6-(2-iminopyrrolidin-1-yl)methyl-2,4(1H,3H)-pyrimidinedione hydrochloride having a purity of at least 90% by mass.

14. The method of claim 1, wherein the nidovirus is a coronavirus.

15. The method of claim 14, wherein the coronavirus is SARS-CoV-2.

16. The method of claim 1, wherein the nidovirus is an arterivirus or torovirus.

17. The method of claim 1, wherein the disease is coronavirus disease (COVID-19), severe acute respiratory syndrome (SARS) virus, Middle East respiratory syndrome (MERS), a respiratory disease, an inflammatory disease, reproductive and respiratory syndrome virus (PRRSV), equine arteritis virus (EAV), or gastroenteritis.

18. A compound of formula (I′): 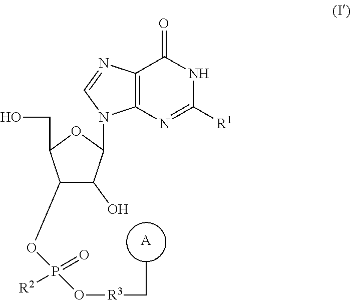
wherein
ring A is 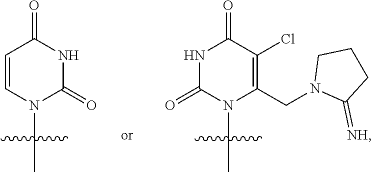 [SMILES: *n1ccc(=O)[nH]c1=O ; *n1c(CN2CCCC2=N)c(Cl)c(=O)[nH]c1=O]
R¹ is —NR⁴R⁵,
R² is O⁻M⁺ or —(CR⁶R⁷)ₙ—R⁸,
R³ is 1′,5′-ribosyl of the formula 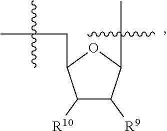
—CH₂CH₂CH₂CH₂—, —CH₂CH₂CH(CO₂H)CH₂—, or —CH₂CH(CO₂H)CH₂CH₂—,
R⁴ and R⁵ is the same or different and each is H or alkyl,
each instance of R⁶ and R⁷ is the same or different and each is H or alkyl,
R⁸ is alkyl, —OR¹¹, —SR¹¹, or —NR⁴R⁵, and
R⁹ and R¹⁰ are the same or different and each is —(CR⁶R⁷)ₘ—R⁸, or R⁹ and R¹⁰ together form —(CR⁶R⁷)ₒ—, and
R¹¹ is H or alkyl,
M⁺ is a counterion,
m and n are the same or different and each is 0 or an integer of 1 to 4, and
is 3 or 4,
provided that the compound of formula (I′) is not GpU dinucleotide.

19. A compound of formula (II′): 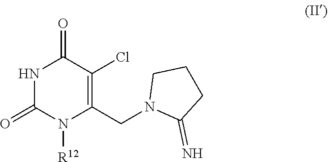
wherein
R¹² is —(CR¹³R¹⁴)ₙ—R⁵,
each instance of R³ and R¹⁴ is the same or different and each is H or alkyl,
R¹⁵ is alkyl, —OR¹⁶, —SR¹⁶, —NR¹⁶R¹⁷, —C(X)OR¹⁶, —C(X)—NR¹⁶R¹⁷, or —NR¹⁶C(X)OR¹⁸,
R¹⁶ and R¹⁸ is the same or different and each is H or alkyl,
R¹⁷ is H, alkyl, OH, or SH,
X is O, S, or NR⁵, and
n is 0 or an integer of 1 to 4,
or a pharmaceutically acceptable salt thereof.

20. The compound of claim 19, wherein R¹³ and R¹⁴ are each H, or a pharmaceutically acceptable salt thereof.

21. The compound of claim 19, wherein R¹⁵ is —OR¹⁶, —NR¹⁶R¹⁷, —C(O)OR¹⁶, —C(O)—NR¹⁶R¹⁷, or —NR¹⁶C(O)OR¹⁸, and n is in integer of 1 to 4, or a pharmaceutically acceptable salt thereof.

22. The compound of claim 21, wherein R¹⁶ is H or methyl, R¹⁷ is H, methyl, or OH, R¹⁸ is methyl, and n is an integer of 2 or 3, or a pharmaceutically acceptable salt thereof.

23. The compound of claim 19, wherein R¹² is —(CH₂)₂OH, —(CH₂)₃OH, —(CH₂)₂OCH₃, —(CH₂)₃OCH₃, —(CH₂)₂NH₂, —(CH₂)₂N(CH₃)₂, —(CH₂)₂CO₂CH₃, —(CH₂)₃CO₂CH₃, —(CH₂)₄CO₂CH₃, —(CH₂)₂NHCO₂H, or —(CH₂)₂NHCO₂CH₃, or a pharmaceutically acceptable salt thereof.
